SEQUENCE LISTING (58 sequences)

SEQ ID NO: 1 - DNA, 517 bp, Saponaria vaccaria
gaccgttaacaatcttgtaatttagtgtgtacaagctctataaatagaggcaagtaatgtggccataaaaggacacacaaaaaacattcaaacaaatcatttaatctctaactttacaagtccaatactttatttgtgaaaatgtctccaatcctcgcccacgacgtagtcaagccccaaggtgtcccagtttgggcttttcaggcaaaagatgttgaaaatgcttcagcccctgtgtaaattaatgtacacaatgcgcttcttcggcctttagatacgatgtttccaaccaaaataaaccataatgttatgtcgagtgtcatgtttcttatttctgtaattttatttctgtatattgtttcgatttttaaattgaaacaataaactatgttaactggtttgtaataaaatctaaaaggccgttctagtgtaaatttaagcattctcctgtcgttcatttctccttagacacattaaaccatactaagataatataattttgaactcaaaatattat

SEQ ID NO: 2 - protein, 32 aa, Saponaria vaccaria
MSPILAHDVVKPQGVPVWAFQAKDVENASAPV

SEQ ID NO: 3 - protein, 6 aa, Saponaria vaccaria
GVPVWA

SEQ ID NO: 4 - DNA, 445 bp, Saponaria vaccaria
gggacagtcggggacacacaaaaaacattcaaacaaatcatttaatctctaactttacaagtccaatactttatttgtgaaaatgtctccaatcctcgcccacgacgtagtcaagccccaaggtgtagcttgggcttttcaggcaaaagatgttgaaaatgcttcagcccctgtgtaaattaatgtacacaatgcgcttcttcggcctttagatacgatgtttccaaccaaaataaaccataatgttatgccgagtgtcatgtttcttatttctgtaattttatttatgtatattgtttcgatttttaaattgaaacaataaactatgttaattggtttgtaataaaatctaaaggccgttctagcgtaaatttaagcattcgcctgtcgttcatttctccaaagacatcattaaaccatactaagataatataattttgaaccc

SEQ ID NO: 5 - protein, 31 aa, Saponaria vaccaria
MSPILAHDVVKPQGVAWAFQAKDVENASAPV

SEQ ID NO: 6 - protein, 31 aa, Saponaria vaccaria
MSPILAHDVVKPQGVAWAFQAKDAENASSPV

SEQ ID NO: 7 - protein, 5 aa, Saponaria vaccaria
GVAWA

SEQ ID NO: 8 - DNA, 365 bp, Saponaria vaccaria
gaatcacacacaaaataaattcatacaaatcatttatttagtctctaacttacaaactccaatacttcatttgtgaaaatgtctccaatttttgcccacgacgtagtcaacccccaaggcctaagtttcgcttttccggcaaaagatgctgaaaatgcttcatccccggtgtaaacttatgtacacaatgcgcttcttcggcctttagatacgatgtttccaaccaaaataaaccataatgttatgtcgagtgtcatgtttcttatttctgtaattttatttctgtatattgtttcgatttttaaattgaaacaataaactatgttaactggtttgtaataaaatctaaaaggccgttctagtac

SEQ ID NO: 9 - protein, 31 aa, Saponaria vaccaria
MSPIFAHDVVNPQGLSFAFPAKDAENASSPV

SEQ ID NO: 10 - DNA, 398 bp, Saponaria vaccaria
aggggaatgacacacaaaataaattcatacaaatcatttatttagtctctaacttacaaactccaatacttcatttgtgaaaatgtctccaatttttgcccacgacgtagtcaagccccaaggcctaagtttcgcttttccggcaaaagatgctgaaaatgcttcatccccggtgtaaacttatgcctgcaatgcgcttctgcggcctttagatacgatgtctccagccaaaccaaaccataatgtcatgtccgacgttgtgtttcttacttttttagttttattttacgtttatcgtttcgacttttaagatgaagaataatgtattttgtttatggtttgtaataaaatttaaaggccgctttagtgtacgtaaatttatggttttgtttccggcc

SEQ ID NO: 11 - protein, 31 aa, Saponaria vaccaria
MSPIFAHDVVKPQGLSFAFPAKDAENASSPV

SEQ ID NO: 12 - protein, 31 aa, Saponaria vaccaria
MSPILAHDVVKPQGLSFAFPAKDAENASSPV

SEQ ID NO: 13 - protein, 5 aa, Saponaria vaccaria
GLSFA

SEQ ID NO: 14 - DNA, 425 bp, Saponaria vaccaria
gctgaaaccacaaattaaagcacaaacataatcaccgataattttacaaacatacatattatcgttcaattcttatcgtacattattattattattgcaagaatggccacctctttccaatttgatggtcttaagccatctttttctgcttcgtacagcagcaagcccattcaaactcaggtttcaaacggcatggacaatgcttctgccccagtgtaaacgcatctagctaatgtccgaaataaatggcctttactagctatagactcgacgtcgagttaataaatcgtatacgatggtgcctcatgtatctcactattgtactcgatcatcaactcgtcgttatgtcatttgtgtgtaatctttataataaaataaataaataaacaaagtcttttggtgagtaagttcaagacttttaactg

SEQ ID NO: 15 - protein, 38 aa, Saponaria vaccaria
MATSFQFDGLKPSFSASYSSKPIQTQVSNGMDNASAPV

SEQ ID NO: 16 - protein, 9 aa, Saponaria vaccaria
FSASYSSKP

SEQ ID NO: 17 - DNA, 395 bp, Saponaria vaccaria
gatgacaacacaaaatacatccaaaaaaattaatttagtctctaacttacaaagtccaaaactactttatttgtgaaaatgtctccaattttcgtccacgaggtggtgaagccccaaggcgtgaaatatgcttttcagccaaaagattctgaaaatgcttcagctccagtgtaaacttacgcatgcaatgcgcttctacggcctttagatacgatgtctccgaccaaaccaaacaataatcttatgtcaagtgttgtattacccgtttctgtaattttattttatgtctattgtttcgacttttaagttgaactatgtaccctaattatgatggtttgtaataaaatttaaaggccattttaatgtacgtaaatttacacatttttcttttgttc

SEQ ID NO: 18 - protein, 31 aa, Saponaria vaccaria
MSPIFVHEVVKPQGVKYAFQPKDSENASAPV

SEQ ID NO: 19 - protein, 5 aa, Saponaria vaccaria
GVKYA

SEQ ID NO: 20 - DNA, 400 bp, Saponaria vaccaria
gatgacgcacaaaaacacatccatacaaatcatttatttagtctttaacttacaaacttcaaaactactttatttgtgaaaatgtctccaatttttgcgcacgacatagtcaagcccaaaggctacagatttagttttcaggcaaaagatgctgaaaatgcttcagccccggtgtaaacttatgtatgcaatgcacttctgcggcttttagatacgatgtctccagccaaatcaaaaaccctaatgtcatatccaatgtcgtgtttcttatttctgtagttttattttatgtttatcgtttcgacttttaagttgaagatgatgtactttgtttatgatttgtaataaaatttaaaagccgtattagtgtacgtaaatttacgattttcttttcgtttaa

SEQ ID NO: 21 - protein, 31 aa, Saponaria vaccaria
MSPIFAHDIVKPKGYRFSFQAKDAENASAPV

SEQ ID NO: 22 - protein, 31 aa, Saponaria vaccaria
MSPIFAHDIVKPKGYRFSFQAKDAENASSPV

SEQ ID NO: 23 - protein, 5 aa, Saponaria vaccaria
GYRFS

SEQ ID NO: 24 - DNA, 360 bp, Saponaria vaccaria
gatcacacaaaacatccaaacaaatcattttagtctcttaacttaattacgtacagtccattactgaaaatgtctccaattttagccctcgacagatacaagcccgaaggccgtgtgaaggcttttcaggcaaaagatgctgaaaatgcttcagccccagtctaaacgtacgtttgcgatgcgtttttgtggtctttagatacgatgcctccaaccaaaccataatgttatgttcaatgttgtgtttcttattttgtaattttattttacgtgtattattttgacttttaaagttgaataatgtacctcgtttatggtttgtaataaaaatctaaaggccattttagtgttacaaaattt

SEQ ID NO: 25 - protein, 31 aa, Saponaria vaccaria
MSPILALDRYKPEGRVKAFQAKDAENASAPV

SEQ ID NO: 26 - protein, 5 aa, Saponaria vaccaria
GRVKA

SEQ ID NO: 27 - DNA, 384 bp, Saponaria vaccaria
gacacacaaaaaacattcaaacaaatcatttaatctctaactttacaagttcaatactttatttgtgaaaatgtctccaatcctctcccacgacgtagtcaagccccaaggtctccctggttggccttttcaggcaaaagatgttgaaaatgcttcagcccctgtgtaaattaatgcacagaatgcgcttcttcggcctttagatacgatgtttccaaccaaaataaaccataatgttatgtcgagtgtcgtgttttttatttctgtaatttatttatgtgtattgtttcaatttttaaattgaaacaataaactattttaattggtttgtaataaaatctaaaaggccgttttagcgtaaatttatgcattcaactgtcgtct

SEQ ID NO: 28 - protein, 32 aa, Saponaria vaccaria
MSPILSHDVVKPQGLPGWPFQAKDVENASAPV

SEQ ID NO: 29 - protein, 6 aa, Saponaria vaccaria
GLPGWP

SEQ ID NO: 30 - DNA, 435 bp, Saponaria vaccaria
aaacctgaaacctcaaacctcaaaccacaaacatatcatatcctatataaattaccgtgaaatcattattattgcgagaatggccacctctttccaacttgatggtcttaagccttcttttggtacgcacggcctgcccgcgccgattcaggttccaaacggcatggacgatgcttgtgccccaatgtagattcatttagcgtctacaataaataaatggcctttactagctttagacttgaagtccccagagtaatattgtgttacgtttagagttgttttattgttgtttacttgcactggacgtcgagttaaatcgtacacgatggtctcttatgtatctcaccactgtacttgataatcaactcctcctcctgtcaattgtgtgtttactttctataagtcaataataaagagtaaaggcatcttttctcc

SEQ ID NO: 31 - protein, 36 aa, Saponaria vaccaria
MATSFQLDGLKPSFGTHGLPAPIQVPNGMDDACAPM

SEQ ID NO: 32 - protein, 9 aa, Saponaria vaccaria
FGTHFLPAP

SEQ ID NO: 33 - DNA, 660 bp, Linum usitatissimum
atggctgctgcttcctctctcgctctggccaccgctagcctagttgctaccggcgccggcggccgtaataacgccttcctaccctcgaagaacaagacaccaaaccttttccttaatcccaacaaaacaacgtcgtcaacagtgaaagctgttgtctcatcatcatcatgcaaacgcccctacccgaaaggagatgctagtttattcttgggtattgatgatgtattcggaaaggatgctgttgctggccatgataatgatcaggatgctgcaagtggccaggagatggccgccgatgatatgttgatgccattcttttggatattcggaaaagaaggacagcagcaggaggccgaggagagcagcgatgatatgttgatgccattcttttggatattcggcaaggaaggacagcagcaggaggccgagagcagcgatgatatgttgctgccattcttttggatattcggcaaggaaggacagcagcaggaggccgagagcagcgatgatatgctgatgcctttcttttggatattcggcaagcagcagcagcagcagggtgagagcagcgatgatatgttgatgcctttcttttgggtattcggcaagcaaggtgacaacaacaagggcgatgctgtagaagcaatccttaagaactag

SEQ ID NO: 34 - DNA, 1602 bp, Linum usitatissimum
atggctgctgcttcctctctcgctctggccaccgctagcctagttgctaccggcgccggcggccgtaataacgccttcctaccctcgaagaacaagacaccaaaccttttccttaatcccaacaaaacaacgtcgtcaacagtgaaagctgttgtctcatcatcatcatgcaaacgcccctacccgaaaggagatgctagtttattcttgggtattgatgatgtattcggaaaggatgctgttgctggccatgataatgatcaggatggtttgttgtttccactcttgctttttatattggggatggcgagaacaaggtgtaggaaattgtttagatatcgtttagatgcatattaactaatcccatcattatatctaactttcttatatctttcttatataaatcaataactttcttatataaatcaataacaaaggtttttagtactaatcaatgattagtatttgctgaagcctttggtttaatgactagtacttgctgaagcctttagattgattacgacttgtgagaatttcatgtgtagcttcttttttcagtttacgctaattggattttggattttctttgtcaatactggctaaaacgtttgatcgaaaaacgatttatcaaagtatttggtaattagggttttcttttaaaagtttttaatggcttcctaattcagttttagataaactattacaactaaccatcaattttggataaactattacaactaaccatcaattttagataaactattacaactaaccatcagttgtagataaactattacaactaaccatcagttgtagataaactattacaactaaccatcagttgtagataaactattacaactaaccatcagttgtagataaactattacaactaaccctctatttatagaatttctcataaactttcaccctatttgaccatcaactcattaagctaatccatttacattaatccggtccatactactaaaaaagtgtgtgtccatattactaaaaaagcgtgtgaaagtgtgtgactttgtaggacccgattcgattagtcgtggtccaaactactaattaacattgacctctaataagatgtgttaactcctaactggaccgaattacttttgattaatcagcctccctagtttttattcggattcggatttaggccgaaggacataaattcttcacaatgatgcagctgcaagtggccaggagatggccgccgatgatatgttgatgccattcttttggatattcggaaaagaaggacagcagcaggaggccgaggagagcagcgatgatatgttgatgccattcttttggatattcggcaaggaaggacagcagcaggaggccgagagcagcgatgatatgttgctgccattcttttggatattcggcaaggaaggacagcagcaggaggccgagagcagcgatgatatgctgatgcctttcttttggatattcggcaagcagcagcagcagcagggtgagagcagcgatgatatgttgatgcctttcttttgggtattcggcaagcaaggtgacaacaacaagggcgatgctgtagaagcaatccttaagaactag

SEQ ID NO: 35 - protein, 219 aa, Linum usitatissimum
MAAASSLALATASLVATGAGGRNNAFLPSKNKTPNLFLNPNKTTSSTVKAVVSSSSCKRPYPKGDASLFLGIDDVFGKDAVAGHDNDQDAASGQEMAADDMLMPFFWIFGKEGQQQEAEESSDDMLMPFFWIFGKEGQQQEAESSDDMLLPFFWIFGKEGQQQEAESSDDMLMPFFWIFGKQQQQQGESSDDMLMPFFWVFGKQGDNNKGDAVEAILKN

SEQ ID NO: 36 - protein, 511 aa, Linum usitatissimum
MAAASSLALATASLVATGAGGRNNAFLPSKNKTPNLFLNPNKTTSSTVKAVVSSSSCKRPYPKGDASLFLGIDDVFGKDAVAGHDNDQDGLLFPLLLFILGMARTRCRKLFRYRLDAYLIPSLYLTFLYLSYINQLSYINQQRFLVLINDYLLKPLVLVLAEAFRLITTCENFMCSFFFQFTLIGFWIFFVNTGNVSKNDLSKYLVIRVFFKFLMASFSFRTITTNHQFWINYYNPSILDKLLQLTISCRTITTNHQLINYYNPSVVDKLLQLTISCRTITTNPLFIEFLINFHPIPSTHANPFTLIRSILLKKCVSILLKKRVKVCDFVGPDSISRGPNYLTLTSNKMCLLTGPNYFLISLPSFYSDSDLGRRTILHNDAAASGQEMAADDMLMPFFWIFGKEGQQQEAEESSDDMLMPFFWIFGKEGQQQEAESSDDMLLPFFWIFGKEGQQQEAESSDDMLMPFFWIFGKQQQQQGESSDDMLMPFFWVFGKQGDNNKGDAVEAILKN

SEQ ID NO: 37 - protein, 8 aa, Linum usitatissimum
MLMPFFWI

SEQ ID NO: 38 - protein, 8 aa, Linum usitatissimum
MLLPFFWI

SEQ ID NO: 39 - protein, 8 aa, Linum usitatissimum
MLMPFFWV

SEQ ID NO: 40 - protein, 48 aa, Citrus paradise
MKTLAGAGMSDPSEGLVLPSSIADDDVGNDNLDLIVIPQYGRNPDYYG

SEQ ID NO: 41 - protein, 6 aa, Citrus paradise
GLVLPS

SEQ ID NO: 42 - protein, 48 aa, Citrus clementina
METTCAGNNWSEGLLLPPFGSIADDDVMNDNLDFLNVPQYGRNPDYMG

SEQ ID NO: 43 - protein, 8 aa, Citrus clementina
GLLLPPFG

SEQ ID NO: 44 - protein, 49 aa, Citrus sinensis
MKTLPGAGMSDPSEGYLLPPSSIADDDVGNDNLDLIVIPQYGRNPDYYG

SEQ ID NO: 45 - protein, 7 aa, Citrus sinensis
GYLLPPS

SEQ ID NO: 46 - protein, 33 aa, Dianthus caryophyllus
MSPNSTRDILKPQGPIPFYGFQAKDAENASVPV

SEQ ID NO: 47 - protein, 7 aa, Dianthus caryophyllus
GPIPFYG

SEQ ID NO: 48 - protein, 32 aa, Dianthus caryophyllus
MSPNSTLDILKPLGLPYEQFQAKDSENASAPV

SEQ ID NO: 49 - protein, 6 aa, Dianthus caryophyllus
GLPYEQ

SEQ ID NO: 50 - protein, 32 aa, Dianthus caryophyllus
MSPNSTRDLLKPLGYKDCCFQAKDLENAAVPV

SEQ ID NO: 51 - protein, 6 aa, Dianthus caryophyllus
GYKDCC

SEQ ID NO: 52 - DNA, 19 bp, Artificial Sequence
  Primer
caccatgtctccaatcctc

SEQ ID NO: 53 - DNA, 18 bp, Artificial Sequence
  Primer
ttacacaggggctgaagc

SEQ ID NO: 54 - DNA, 21 bp, Artificial Sequence
  Primer
ccgacagtggtcccaaagatg

SEQ ID NO: 55 - DNA, 20 bp, Artificial Sequence
  Primer
gcctgaaaagcccaaactgg

SEQ ID NO: 56 - DNA, 32 bp, Artificial Sequence
  Primer
gcggccgcatggctgctgcttcctctctcgct

SEQ ID NO: 57 - DNA, 38 bp, Artificial Sequence
  Primer
cctgcaggctagttcttaaggattgcttctacagcatc

SEQ ID NO: 58 - DNA, 38 bp, Artificial Sequence
  Linker sequence
gcggccgcaaaaaacctgcaggacccgggaggcgcgcc

(19) United States
(12) Patent Application Publication
(10) Pub. No.: US 2012/0058905 A1
(43) Pub. Date: Mar. 8, 2012

(54) DNA Sequences Encoding Caryophyllaceae and Caryophyllaceae-Like Cyclopeptide Precursors and Methods of Use

(76) Inventors: Patrick S. Covello, Saskatoon (CA); Raju S.S. Datla, Saskatoon (CA); Sandra Lee Stone, Saskatoon (CA); J. John Balsevich, Saskatoon (CA); Martin John Reaney, Saskatoon (CA); Paul Grenville Arnison, Saskatoon (CA); Janet Anne Condie, Saskatoon (CA)

(21) Appl. No.: 13/319,697

(22) PCT Filed: May 10, 2010
(86) PCT No.: PCT/CA10/00700

§ 371 Date: Nov. 10, 2011

Related U.S. Application Data
(60) Provisional application No. 61/213,198, filed on May 15, 2009.

(51) Int. Cl. C40B 30/02; C07K 5/062; C12N 15/63; C07K 14/00; C12N 5/10; C07H 21/04

(52) U.S. Cl. 506/8; 530/321; 530/402; 435/419; 536/23.1; 435/320.1; 530/324; 562/559; 530/350

(57) ABSTRACT

Naturally-occurring and modified recombinant nucleic acid molecules have been isolated that encode linear pre-cursors of cyclopeptides of the Caryophyllaceae (Ccps) and Caryophyllaceae-like (Clcps) type V1 class of cyclopeptides. Such nucleic acid molecules are useful for producing cyclopeptides and their linear precursors by recombinant methods.

### CROSS-REFERENCE TO RELATED APPLICATIONS

This application claims the benefit of U.S. Provisional Patent Application Ser. No. 61/213,198 filed May 15, 2009, the entire contents of which are herein incorporated by reference.

### FIELD OF THE INVENTION

The present invention relates to nucleic acid molecules encoding cyclopeptide precursors, to the cyclopeptide precursors encoded by the nucleic acids, to cyclopeptides formed from the precursors, and to methods of use thereof.

### BACKGROUND OF THE INVENTION

More than 450 naturally-occurring higher plant cyclopeptides, from 26 families, 65 genera and 120 species have been described (Tan 2006). On the basis of structure and phylogentic distribution the authors have proposed a systematic structural classification of plant cyclopeptides which is divided into two classes, five sub-classes and eight types.

According to the skeletons, whether formed with amino acid peptide bonds or not, cyclopeptides can be divided into two classes, i.e., heterocyclopeptides and homocyclopeptides. Then on the basis of the number of rings, these classes can be divided into five subclasses, i.e., heteromonocyclopeptides, heterodicyclopeptides, homomonocyclopeptides, homodicyclopeptides, and homopolycyclopeptides. Finally, according to the characteristics of rings and sources, cyclopeptides can be divided into eight types. The numbers of cyclopeptides discovered from higher plants up to 2005, which belong to types I, II, III, IV, V, VI, VII, and VIII are 185, 2, 4, 13, 9, 168, 23, and 51, respectively. Among them, types I and VI are the largest two types. These 455 cyclopeptides involve cyclic di- (2), tri- (3), tetra- (4), penta- (5), hexa- (6), hepta- (7), octa- (8), nona- (9), deca- (10), undeca- (11), dodeca- (12), tetradeca- (14), octacosa-(28), nonacosa- (29), traconta- (30), hentriaconta- (31), tetratraconta- (34), and heptatraconta- (37) peptides, respectively.

Other classification schemes for cyclopeptides from diverse origins have been described based on ring size for example (Davies 1999).

Regarding the naturally occurring cyclopeptides described of plant origin only the cyclotides, group VII (Tan 2006) are currently known to have a genetic basis for synthesis wherein a gene encoding a linear peptide precursor produced by ribosomal synthesis is cyclized by the recruitment of endogenous proteolytic enzymes (Gruber 2008).

Many different cyclopeptides have been described from natural sources, in addition to those of plant origin, that have been of great interest as many have important biological functions, especially as antibiotics. It is noteworthy that the largest majority of such cyclopeptides are also made by non-ribosomal synthesis involving large protein complexes, (NRPS), (Seiber 2003, Grunewald 2006). An exception is a family of cyclopeptides exemplified by patellamides isolated from ascidians with obligate cyanobacterial sympionts identified as Prochloron spp. (Donia 2006).

The Caryophyllaceae (the Pink or Carnation family) and Caryophyllaceae-like cyclopeptides belong to class VI (Tan 2006) include known cyclo di, penta, hexa, hepta, octo, nona, dedca, undeca and dodeca cyclopeptides.

Ccps are known from the Caryophyllaceae genera: Arenaria, Brachystemma, Cerastium, Dianthus, Drymania, Polycarpon, Psammosilene, Pseudostellaria, Silene, Stellaria, and Saponaria (=Vaccaria)

Clcps are known from families genetically related to the Caryophyllaceae such as: Annonaceae, Araliaceae, (e.g. genus Panax), Euphorbiaceae, (e.g. genus Jatropha), Labiatae, Linaceae, (e.g. genus Linum), Phytolaccaceae, Rutaceae, (e.g. genus Citrus), and Vebebaceae.

Cyclopeptides are known bioactive compounds with wide pharmacological properties (Sarabia 2004, Craik 2004).

Naturally occurring cyclopeptides from Saponaria vaccaria, (=Vaccaria segetalis), Citrus natsudaidai and other species are known to possess vasodilatory activity, (Morita 2006, Morita 2007). Additionally, the segetalins from Saponaria vaccaria are reported to possess estrogen-like activity (Morita 1995a, Morita 1997, Yun 1997) and growth inhibitory and antihelmintic activity (Morita 1996; Dahiya 2007a, Dahiya 2007b).

The naturally-occurring cyclopeptides from flax are known to have strong immunosuppressive, and anti-malarial activity (Picur 2007).

The wide variation in bioactivity and utility of cyclopeptides is confirmed by many studies and patents directed to synthetically produced peptides. Examples include, but are not limited to: anti-bacterial activity (U.S. Pat. No. RE39,071, U.S. Pat. No. 7,153,826, U.S. Pat. No. 6,890,537); anti-fungal activity (U.S. Pat. No. 7,015,309); anti-biotic activity (U.S. Pat. No. 7,169,756); anti-protozoan activity (U.S. Pat. No. 5,957,837); anti-viral activity (U.S. Pat. No. 6,943,233); anti-cancer activity (U.S. Pat. No. 7,138,369, U.S. Pat. Nos. 7,122,623, 7,199,100); hormone analog activity (U.S. Pat. No. 7,144,859, U.S. Pat No. 7,018,981); and, inhibition of enzymes (U.S. Pat. No. 7,045,504).

### SUMMARY OF THE INVENTION

The present invention provides naturally-occurring and modified recombinant nucleic acid molecules encoding linear polypeptide precursors of cyclopeptides of the Caryophyllaceae (Ccps) and Caryophyllaceae-like (Clcps) type VI class of cyclopeptides as defined in Plant Cyclopeptides (Tan 2006).

The invention also provides a recombinant chimeric gene construct, encoding linear polypeptide precursors of all or part of the plant Ccp or Clcp cyclopeptides, wherein expression of said recombinant chimeric gene results in the production of Ccp or Clcp cyclopeptides, linear polypeptide precursors of Ccp or Clcp cyclopeptides or linear polypeptide precursors of modified Ccp or Clcp cyclopeptides in a transformed host cell.

The invention additionally provides the recovery and purification of cyclopeptides of the Caryophyllaceae (Ccps) and Caryophyllaceae-like (Clcps) from plant material.

Embodiments of the present invention are directed to cyclizable molecules and their linear precursors; cyclopeptides or derivative forms of the cyclized molecules and their linear precursors encoded by the subject nucleic acid molecules. The cyclic and linear peptides, polypeptides or proteins may be naturally occurring or may be modified by the insertion or substitution of heterologous amino acid sequences.

The embodiments of the present invention are further directed to conserved nucleotide flanking sequences of nucleic acid molecules that encode cyclopeptides. The flanking sequences encode regions of linear polypeptides that provide for the cyclization of polypeptides that are encoded between the flanking sequences.

One embodiment of the present invention provides isolated nucleic acid molecules, derived from Saponaria vaccaria, comprising a sequence of nucleotides, which sequence of nucleotides, or its complementary form, encodes an amino acid sequence or a derivative form thereof capable of being cyclized within a cell to form known segetalin A, B, C, D, E, F, G and H.

A further embodiment of the present invention provides isolated DNA sequences, derived from Linum usitatissimum, comprising a sequence of nucleotides, which sequence of nucleotides, or its complementary form, encodes an amino acid sequence or a derivative form thereof capable of being cyclized within a cell to form known cyclolinopeptides D, F, G or H.

A further embodiment of the present invention provides for isolated nucleic acid molecules, derived from Saponaria vaccaria comprising a sequence of nucleotides, which sequence of nucleotides, or its complementary form, encodes an amino acid sequence or a derivative form thereof capable of being cyclized within a cell to form segetalin cyclopeptides that have not yet been chemically detected and characterized.

A further embodiment of the present invention provides for discovery of nucleic acid molecules, derived from species within the Caryophyllaceae and genetic related families, which sequences or their complementary forms, encode an amino acid sequence or a derivative form thereof capable of being cyclized within a cell to form Caryophyllaceae (Ccps) and Caryophyllaceae-like (Clcps) type VI class of cyclopeptides. Said Caryophyllaceae (Ccps) and Caryophyllaceae-like (Clcps) type VI class cyclopeptides may not have been previously chemically detected and characterized.

The embodiments comprise a peptide sequence that can be processed from a larger polypeptide sequence from any member of the Caryophyllaceae and genetically related families comprising Caryophyllaceae (Ccps) and Caryophyllaceae-like (Clcps) type VI class of cyclopeptides. More specifically, the embodiments refer to a peptide sequence, derived from Saponaria vaccaria or Linum usitatissimum which can be cleaved and cyclized. The embodiments further extend to linear forms and precursor forms of the peptide, polypeptide or protein, which may also have activity or other utilities. The embodiments additionally extend to engineering genetically unrelated plants with the sequences of the embodiments in order to produce plants that have added value, improved agronomic performance or serve as a host for the production and subsequent recovery of said cyclized peptide sequence.

The embodiments further extend to a method of producing a cyclopeptide comprising: transforming a host cell, tissue or organism with means for encoding a linear polypeptide to thereby produce the linear polypeptide in the cell, tissue or organism; and, cyclizing the linear polypeptide to produce the cyclopeptide.

The embodiments further extend to engineering a microorganism such as a bacterium, yeast or fungus to express a peptide sequence derived from any member of the Caryophyllaceae and genetic related families comprising Caryophyllaceae (Ccps) and Caryophyllaceae-like (Clcps) type VI class of cyclopeptides. More specifically, the embodiments refer to a peptide sequence, which can be cleaved and cyclized. The embodiments further extend to linear forms and precursor forms of the peptide, polypeptide or protein, which may be recovered and also have activity or other utilities. More specifically the embodiments extend to a peptide sequence from Saponaria vaccaria or Linum usitatissimum that can be processed from a larger polypeptide sequence to produce Caryophyllaceae (Ccps) and Caryophyllaceae-like (Clcps) type VI class of cyclopeptides.

A further embodiment of the present invention provides an isolated nucleic acid molecule comprising a sequence of nucleotides, which sequence of nucleotides, or its complementary form, encodes an amino acid sequence or a derivative form thereof capable of forming a structural homologue of a cyclopeptide within a cell, more specifically a structural homolog of a Caryophyllaceae (Ccps) and Caryophyllaceae-like, (Clcps) type VI class of cyclopeptides.

The embodiments include an isolated nucleic acid molecule comprising a nucleotide sequence having at least 80% sequence identity to the nucleotide sequence as set forth in SEQ ID NO: 1, SEQ ID NO: 4, SEQ ID NO: 8, SEQ ID NO: 10, SEQ ID NO: 14, SEQ ID NO: 17, SEQ ID NO: 20, SEQ ID NO: 24, SEQ ID NO: 27, SEQ ID NO: 30, SEQ ID NO: 33 or SEQ ID NO: 34, or a full length complement thereof.

The embodiments further include an isolated nucleic acid molecule comprising the nucleotide sequence flanking a cyclopeptide encoding region of the nucleotide sequences as set forth in SEQ ID NO: 1, SEQ ID NO: 4, SEQ ID NO: 8, SEQ ID NO: 10, SEQ ID NO: 14, SEQ ID NO: 17, SEQ ID NO: 20, SEQ ID NO: 24, SEQ ID NO: 27, SEQ ID NO: 30, SEQ ID NO: 33 or SEQ ID NO: 34.

The embodiments further include a nucleic acid construct comprising one or more of the nucleic acid molecules of the present invention operatively linked to one or more nucleotide sequences for aiding in transformation of a cell with the construct. The embodiments also relate to a chimeric gene construct comprising an isolated polynucleotide of the embodiments operably linked to suitable regulatory sequence. A further embodiment concerns an isolated host cell comprising a chimeric gene construct or an isolated polynucleotide of the embodiments. The host cell may be eukaryotic, such as a yeast or a plant cell, or prokaryotic, such as a bacterial cell. The embodiments also relate to a virus comprising a chimeric gene construct or an isolated polynucleotide of the embodiments. The embodiments further provide a process for producing an isolated host cell comprising a chimeric gene construct or an isolated polynucleotide of the embodiments, the process comprising either transforming or transfecting an isolated compatible host cell with a chimeric gene construct or an isolated polynucleotide of the embodiments.

The embodiments further include an isolated linear polypeptide comprising the amino acid sequence a set forth in SEQ ID NO: 2, SEQ ID NO: 5, SEQ ID NO: 6, SEQ ID NO: 9, SEQ ID NO: 11, SEQ ID NO: 12, SEQ ID NO: 15, SEQ ID NO: 18, SEQ ID NO: 21, SEQ ID NO: 22, SEQ ID NO: 25, SEQ ID NO: 28, SEQ ID NO: 31, SEQ ID NO: 35 or SEQ ID No: 36.

The embodiments further include an isolated cyclopeptide consisting of the amino acid sequence as set forth in SEQ ID NO: 26, SEQ ID NO: 29, SEQ ID NO: 32, SEQ ID NO: 37, SEQ ID NO: 38, SEQ ID NO: 39, SEQ ID NO: 41, SEQ ID NO: 43, SEQ ID NO: 45, SEQ ID NO: 47, SEQ ID NO: 49 or SEQ ID NO: 51.

The embodiments further include a method of producing a cyclopeptide comprising: providing a linear polypeptide comprising the amino acid sequence as set forth in SEQ ID NO: 3, SEQ ID NO: 7, SEQ ID NO: 13, SEQ ID NO: 16, SEQ ID NO: 19, SEQ ID NO: 23, SEQ ID NO: 26, SEQ ID NO: 29, SEQ ID NO: 32, SEQ ID NO: 37, SEQ ID NO: 38, SEQ ID NO: 39, SEQ ID NO: 40, SEQ ID NO: 43, SEQ ID NO: 45, SEQ ID NO: 47, SEQ ID NO: 49 or SEQ ID NO: 51; and, subjecting the linear polypeptide to conditions under which a cyclopeptide consisting of the amino acid sequence as set forth in SEQ ID NO: 3, SEQ ID NO: 7, SEQ ID NO: 13, SEQ ID NO: 16, SEQ ID NO: 19, SEQ ID NO: 23, SEQ ID NO: 26, SEQ ID NO: 29, SEQ ID NO: 32, SEQ ID NO: 37, SEQ ID NO: 38, SEQ ID NO: 39, SEQ ID NO: 41, SEQ ID NO: 43, SEQ ID NO: 45, SEQ ID NO: 47, SEQ ID NO: 49 or SEQ ID NO: 51 is produced by cyclization of the linear polypeptide.

A still further embodiment of the inventions provides a method to discover DNA sequences that encode Caryophyllaceae, (Ccps) and Caryophyllaceae-like, (Clcps) type VI class of cyclopeptides, using conserved flanking DNA sequences of known cyclopeptide encoding sequences as a probe. This embodiment is particularly useful for the identification of DNA sequences that encode cyclopeptides of small size that could not be identified conveniently by conventional means. Thus, the embodiments further include a method of identifying a gene or polypeptide related to cyclopeptide production comprising: selecting a nucleic acid molecule that is known to encode a reference cyclopeptide; identifying a flanking sequence in the nucleic acid molecule or in a linear polypeptide encoded by the nucleic acid molecule, the flanking sequence flanking a nucleotide sequence of the nucleic acid molecule that encodes the reference cyclopeptide or flanking an amino acid sequence of the linear polypeptide that corresponds to the reference cyclopeptide; searching a database of nucleic acid molecules or polypeptides for target sequences that have at least 80% sequence identity to the flanking sequence to thereby identify nucleotide or amino acid sequences that correspond to the gene or polypeptide related to cyclopeptide production.

The embodiments further include a method of identifying a gene or polypeptide related to cyclopeptide production comprising: generating a database of amino acid sequences from translation of known nucleotide sequences for an organism; and, searching the database of amino acid sequences for exact matches with all circular permutations of a known cyclic peptide from the organism to identify nucleotide sequences that correspond to a gene in the organism which encodes the polypeptide related to cyclopeptide production.

A further embodiment of the invention provides a method to recover, separate and purify to homogeneity cyclopeptides. In particular, the invention provides for a method to recover and separate cyclopeptides A, B and D, extracted from seed of Saponaria vaccaria. In particular, the invention provides for a method to recover and purify to homogeneity cyclopeptide A from seed of Saponaria vaccaria cv Pink Beauty. The embodiment further includes a method of producing a cyclopeptide comprising providing a dry extract of a plant tissue containing the cyclopeptide, dissolving the extract in a solvent comprising at least 90% ethanol to form a cyclopeptide-rich solution; and recovering the cyclopeptide from the solution.

The embodiments further include a method of reducing cyclopeptide content in a host cell, tissue or plant comprising: reducing expression in the cell, tissue or plant of a nucleic acid molecule comprising a nucleotide sequence having at least 80% sequence identity to the nucleotide sequence as set forth in SEQ ID NO: 1, SEQ ID NO: 4, SEQ ID NO: 8, SEQ ID NO: 10, SEQ ID NO: 14, SEQ ID NO: 17, SEQ ID NO: 20, SEQ ID NO: 24, SEQ ID NO: 27, SEQ ID NO: 30, SEQ ID NO: 33 or SEQ ID NO: 34, compared to expression of the nucleotide sequence in the cell, tissue or plant before expression was reduced.

Further features of the invention will be described or will become apparent in the course of the following detailed description.

### BRIEF DESCRIPTION OF THE DRAWINGS

In order that the invention may be more clearly understood, embodiments thereof will now be described in detail by way of example, with reference to the accompanying drawings, in which:

FIG. 1 depicts a comparison of predicted amino acid sequences based on segetalin precursor gene sequences. Manual alignment of predicted amino acid sequences of cDNAs encoding putative segetalin precursors of S. vaccaria is shown. Known and predicted mature cyclic peptide sequences are shown in reverse type. Amino acid positions showing complete conservation are highlighted in gray.

FIG. 2 depicts LC/MS analysis of hairy root samples. Expression of presegetalin A results in segetalin A formation in transformed roots of S. vaccaria, White Beauty. Single ion chromatograms (m/z 610, M+1 in ESI+mode) are shown for A, segetalin A standard; B, C and D, three independent hairy root lines expressing sgala; E, hairy root line pK7-OE-9 (control); and F, a control hairy root line derived from wild type A. rhizogenes LBA9402.

FIG. 3 depicts mass spectrophotometric analysis of segetalin A showing fragment ions under ES⁺ conditions showing M+1 (m/z 610) and fragment ions m/z 582 and m/z 511 that were used to verify presence of segetalin A in hairy root samples.

FIG. 4 depicts production of segetalin A in transformed S. vaccaria white beauty transformed hairy root cultures. Hairy root cultures were generated using A. rhizogenes harbouring pJC003 (for presegetalin A expression) or pK7WG2D (empty vector, denoted by pK7-OE). Plasmid and root culture lines are indicated. Segetalin A was determined by LC/MS using triplicate samples. Means and standard deviations are indicated.

FIG. 5A depicts a diagram of the extraction procedure for segetalins from S. vaccaria showing separation of cyclopeptide-containing fraction CPs A,B,D+ from the methanol extract of Saponaria seed.

FIG. 5B depicts a chromatogram of a cyclic peptide-containing fraction showing a mixture of known segetalins A, B and D.

FIG. 6A depicts Flax (Bethune) CP1 genomic sequence (1602 bp) with exons highlighted gray.

FIG. 6B depicts CP1 amino acid sequence (219 aa) with cyclopeptide sequences bold and underlined.

FIG. 6C depicts CP1 genomic sequence translated with exons highlighted in gray. Five cyclic peptide sequences shown in bold and underlined occur in the 2nd exon.

FIG. 7 depicts SDS-PAGE analysis of GST-CP1 precursor protein expression induced in E. coli cells after 3 h of arabinose treatment (+).

FIG. 8 depicts a map of d35S:CP1 cDNA expression vector.

FIG. 9 depicts a graph showing that d35S:CP1 cDNA expression increases specific cyclic peptide levels found in wild type Normandy flax seeds. LC MS areas calculated for the five cyclic peptide forms encoded by CP1 cDNA in extracts of wild type Normandy seeds and d35S:CP1 cDNA T1 seeds. Black arrows indicate cyclic peptide forms that show increased levels in the two independent transgenic lines.

### DESCRIPTION OF PREFERRED EMBODIMENTS

### Terms

In order to facilitate review of the various embodiments of the disclosure, the following explanations of specific terms are provided:

Complementary nucleotide sequence: “Complementary nucleotide sequence” of a sequence is understood as meaning any DNA whose nucleotides are complementary to those of sequence of the disclosure, and whose orientation is reversed (antiparallel sequence).

Degree or percentage of sequence homology: The term “degree or percentage of sequence homology” refers to degree or percentage of sequence identity between two sequences after optimal alignment. Percentage of sequence identity (or degree or identity) is determined by comparing two optimally aligned sequences over a comparison window, where the portion of the peptide or polynucleotide sequence in the comparison window may comprise additions or deletions (i.e., gaps) as compared to the reference sequence (which does not comprise additions or deletions) for optimal alignment of the two sequences. The percentage is calculated by determining the number of positions at which the identical amino-acid residue or nucleic acid base occurs in both sequences to yield the number of matched positions, dividing the number of matched positions by the total number of positions in the window of comparison and multiplying the result by 100 to yield the percentage of sequence identity.

Isolated: As will be appreciated by one of skill in the art, “isolated” refers to polypeptides or nucleic acids that have been “isolated” from their native environment.

Nucleotide, polynucleotide, or nucleic acid sequence: “Nucleotide, polynucleotide, or nucleic acid sequence” will be understood as meaning both a double-stranded or single-stranded DNA in the monomeric and dimeric (so-called in tandem) forms and the transcription products of said DNAs.

Sequence identity: Two amino-acid or nucleotide sequences are said to be “identical” if the sequence of amino-acids or nucleotide residues in the two sequences is the same when aligned for maximum correspondence as described below. Sequence comparisons between two (or more) peptides or polynucleotides are typically performed by comparing sequences of two optimally aligned sequences over a segment or “comparison window” to identify and compare local regions of sequence similarity. Optimal alignment of sequences for comparison may be conducted by the local homology algorithm of Smith and Waterman (Smith 1981), by the homology alignment algorithm of Neddleman and Wunsch (Neddleman 1970), by the search for similarity method of Pearson and Lipman (Pearson 1988), by computerized implementation of these algorithms (GAP, BESTFIT, FASTA, and TFASTA in the Wisconsin Genetics Software Package, Genetics Computer Group (GCG), 575 Science Dr., Madison, Wis.), or by visual inspection. Isolated and/or purified sequences of the present invention or used in the present invention may have a percentage identity with the bases of a nucleotide sequence, or the amino acids of a polypeptide sequence, of at least about 80%, 81%, 82%, 83%, 84%, 85%, 86%, 87%, 88%, 89%, 90%, 91%, 92%, 93%, 94%, 95%, 96%, 97%, 98%, 99%, 99.5%, 99.6%, or 99.7%. This percentage is purely statistical, and it is possible to distribute the differences between the two nucleotide sequences at random and over the whole of their length.

It will be appreciated that this disclosure embraces the degeneracy of codon usage as would be understood by one of ordinary skill in the art and as illustrated in Table 1. Furthermore, it will be understood by one skilled in the art that conservative substitutions may be made in the amino acid sequence of a polypeptide without disrupting the structure or function of the polypeptide. Conservative substitutions are accomplished by the skilled artisan by substituting amino acids with similar hydrophobicity, polarity, and R-chain length for one another. Additionally, by comparing aligned sequences of homologous proteins from different species, conservative substitutions may be identified by locating amino acid residues that have been mutated between species without altering the basic functions of the encoded proteins. Table 2 provides an exemplary list of conservative substitutions.

TABLE 1
Codon Degeneracies

| Amino Acid | Codons |
|---|---|
| Ala/A | GCT, GCC, GCA, GCG |
| Arg/R | CGT, CGC, CGA, CGG, AGA, AGG |
| Asn/N | AAT, AAC |
| Asp/D | GAT, GAC |
| Cys/C | TGT, UGC |
| Gln/Q | CAA, CAG |
| Glu/E | GAA, GAG |
| Gly/G | GGT, GGC, GGA, GGG |
| His/H | CAT, CAC |
| Ile/I | ATT, ATC, ATA |
| Leu/L | TTA, TTG, CTT, CTC, CTA, CTG |
| Lys/K | AAA, AAG |
| Met/M | ATG |
| Phe/F | TTT, TTC |
| Pro/P | CCT, CCC, CCA, CCG |
| Ser/S | TCT, TCC, TCA, TCG, AGT, AGC |
| Thr/T | ACT, ACC, ACA, ACG |
| Trp/W | TGG |
| Tyr/Y | TAT, TAC |
| Val/V | GTT, GTC, GTA, GTG |
| START | ATG |
| STOP | TAG, TGA, TAA |

TABLE 2
Conservative Substitutions

| Type of Amino Acid | Substitutable Amino Acids |
|---|---|
| Hydrophilic | Ala, Pro, Gly, Glu, Asp, Gln, Asn, Ser, Thr |
| Sulphydryl | Cys |
| Aliphatic | Val, Ile, Leu, Met |
| Basic | Lys, Arg, His |
| Aromatic | Phe, Tyr, Trp |

The definition of sequence identity given above is the definition that would be used by one of skill in the art. The definition by itself does not need the help of any algorithm, said algorithms being helpful only to achieve the optimal alignments of sequences, rather than the calculation of sequence identity. From the definition given above, it follows that there is a well defined and only one value for the sequence identity between two compared sequences which value corresponds to the value obtained for the best or optimal alignment. In the BLAST N or BLAST P “BLAST 2 sequence”, software which is available in the web site http://www.ncbi.nlm.nih.gov/gorf/bl2.html, and habitually used by the inventors and in general by the skilled man for comparing and determining the identity between two sequences, gap cost which depends on the sequence length to be compared is directly selected by the software (i.e. 11.2 for substitution matrix BLOSUM-62 for length>85).

### Cyclopeptides

Cyclopeptides derived from natural sources have been classified in several ways, however the majority of such plant peptide classes, with the notable exception of large peptides known as cyclotides (Gruber 2008) are formed by large protein complexes. However, until the present invention, it was not known that cyclopeptides made by plants of the Caryophyllaceae (Ccps) and Caryophyllaceae-like (Clops) genetically related genera were encoded by genes and are manufactured by ribosomes.

The potential therapeutic value of such cyclopeptides has motivated the chemical synthesis of one form of Saponaria cyclopeptide, (segetalin C) (Dahiya 2008a) and a cyclopeptide from the peel of Citrus (Dahiya 2008b). Cyclopeptides are considered of significant commercial potential for medicinal and therapeutic purposes because of their chemical nature.

Cyclopeptides derived from the Caryophyllaceae and related plant families are produced by the cyclization of linear precusor proteins and have the carboxy and amino terminal groups joined. Peptide cyclization rigidifies structure and improves in vivo stability of small bioactive molecules. A variety of chemical strategies have been described for the cyclization of linear peptide molecules (Davies 2007). Additionally, cyclization can be achieved using self splicing proteins called inteins. Inteins excise themselves from a precursor protein (Scott 1999).

In the present invention, an indication that segetalins and cyclopeptides from related species were encoded by genes was indicated by the occurrence of different cyclopeptides amongst wild type and cultivated forms of Saponaria vaccaria. Varieties had both unique profiles and differing amounts of individual cyclopeptides (see Table 3). Table 3 describes the occurrence and relative abundance of cyclopeptides present in the seed of different accessions and wild types of Saponaria vaccaria.

TABLE 3
Segetalin profiles from different accessions and wild types

| Segetalin | MW | PB | UM | BT-WBLX | TURK | SCOTT | FINL | WB | MONG |
|---|---|---|---|---|---|---|---|---|---|
| A | 609.3 | +++ | +++ | +++ | +++ | ++++ | +++ | − | +++ |
| B | 484.2 | ++ | ++ | ++ | + | + | + | ++ | ++ |
| C | 769.4 | − | − | − | − | − | − | − | − |
| D | 719.4 | +++ | ++ | ++ | + | ++ | + | + | − |
| E | 812.4 | − | − | − | − | − | − | − | + |
| F | 954.4 | +++ | +++ | +++ | ++ | ++ | ++ | ++ | + |
| G | 518.3 | +++ | +++ | +++ | − | − | − | ++ | +++ |
| H | 610.3 | + | + | ++ | ++ | ++ | ++ | + + | + |

PB—Pink Beauty, obtained from CN seeds Ltd, Pymoor, Ely, Cambrdgeshire, UK.
UM—Cowcockle wild type from University of Manitoba.
BT-WBLX—Vaccarria sp (wang bu liu xing), B and T World Seeds, Paguignan, France.
TURK—Wild type Vaccaria hispanica, Accession PI 304488 with origin in Turkey, obtained from North Central Regional Plant Introduction Station, USDA-ARS.
SCOTT—Land race developed by Agriculture and Agri-Food Canada by recurrent selection of wild type cowcockle.
FINLAND—Wild type Vaccaria hispanica, Accession PI 578121 with origin in Finland, obtained from North Central Regional Plant Introduction Station, USDA-ARS.
WB—White Beauty, accession obtained from CN seeds Ltd, Pymoor, Ely, Cambrdgeshire, UK.
MONG—Wild type Vaccaria hispanica, Accession PI 597629 with origin in Mongolia, obtained from North Central Regional Plant Introduction Station, USDA-ARS.

PB, UM and BT-WBLX have similar CP profiles. TURK, SCOTT, and FINL have similar CP profiles (but different saponin profiles). All three varieties have no segetalin G. WB and MONG are unique. WB has no segetalin A. MONG has no segetalin D and is the only collected material with segetalin E. No segetalin C was observed in any of these collections but has been reported in the literature (Morita 1995b) and synthesized (Gruber 2008).

Further evidence for the apparent segregation and differing expression of segetalin genes was obtained from the analysis of doubled haploid lines derived form Pink Beauty, White Beauty and crosses between these accessions and land race Scott. Doubled haploid lines were produced by known methods (Ferrie 2006).

One method to determine the presence of expressed genes in an organism is to prepare a library of expressed sequence tags that correspond to the genes that are expressed in cells. An expressed sequence tag or EST is a short sub-sequence of a messenger RNA (mRNA). ESTs are used to identify gene transcripts and determine gene sequences. An EST is produced by sequencing a small number to several hundred base pairs from the end of a cDNA clone taken from a cDNA library. Because these clones consist of DNA that is complimentary to mRNA, the ESTs represent portions of expressed genes.

ESTs prepared from any species in the Caryophyllaceae family or genetically related families comprising cyclopeptides of the Caryophyllaceae (Ccps) and Caryophyllaceae-like (Clops) type VI class of cyclopeptides can be used to identify gene sequences containing coding sequences for linear precursor proteins that can be cyclized to form the cyclopeptides. This is true for cyclopeptides that are known from the literature and have been chemically characterized such that the DNA sequences can be predicted from known peptide sequences. This is additionally true for cyclopeptides that have not yet been discovered or chemically characterized, or are too small to be identified by other methods, e.g. by using conserved cyclopeptide cyclizing flanking sequences as a probe.

Cyclopeptides derived from many natural sources are well known for bioactivity and thus it would be apparent that cyclopeptides derived from the Caryophyllaceae and genetically related families will also possess such activities that can be determined by known methods in the art.

It is anticipated that the natural function of plant cyclopeptides is in relation to the protection of plants from natural predation from, for example, insects or other herbivores and from disease causing organisms such as viruses, bacteria and fungi. It is apparent that an indication of the natural function of the Caryophyllaceae (Ccps) and Caryophyllaceae-like (Clcps) type VI class of cyclopeptides can be evaluated by searching databases of known DNA sequences, (i.e. GenBank), using known search engines to identify related sequences where the function of said sequences is known.

### Expression

Therefore, it is evident that DNA sequences for cyclopeptides derived form the Caryolphyllaceae and genetically related families can be expressed in alternate plant hosts to impart characteristics of improved agronomic performance via recombinant means. The methods to construct DNA expression vector and to transform and express foreign genes in plant and plant cells are well known in the art.

It is additionally evident that such heterologous expression can be conducted in microorganisms, such as in bacteria, yeast and in fungi, which can this serve as host for the recombinant expression, production and isolation of cyclopeptides for diverse purposes that include but are not limited to: medical and therapeutic purposes as drugs for the treatment of disease and other medical conditions.

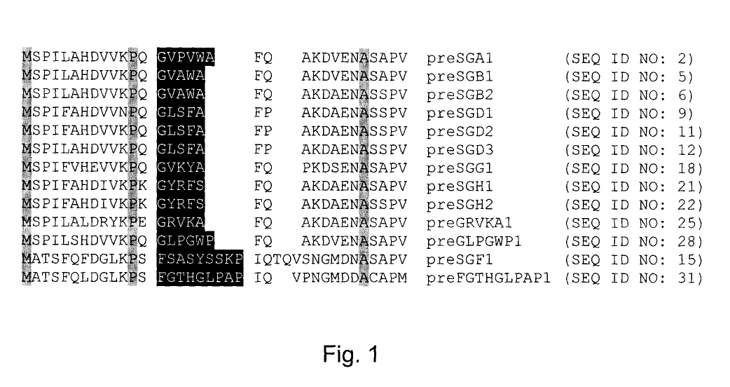

It is apparent from examination of the sequences of the precursor proteins for cyclopeptide formation in Saponaria vaccaria (FIG. 1) that the flanking sequences that surround the cyclopeptide sequences are highly conserved and display only minor variation, whereas the sequences of the cyclopeptides themselves are highly variable. The conservation of flanking sequences suggests that these sequences are highly relevant for the cyclization reaction, whether such cyclization is the result of spontaneous cyclization or the result of enzymatic cyclization. Further, high conservation of the flanking sequences provides for the ability to use the flanking sequences to probe for hitherto unknown gene and polypeptide sequences involved in the production of cyclopeptides.

Additionally, it is evident that the sequences can be used in the construction of an expression vector for the cyclization of peptides contained within said cyclization sequences. It is well known that DNA sequences encoding cyclopeptides can be inserted within an expression vector for heterologous expression in diverse host cells and organisms, for example plant cells and plant, by conventional techniques. These methods, which can be used in the invention, have been described elsewhere (Potrykus 1991; Vasil 1994; Walden 1995; Songstad 1995), and are well known to persons skilled in the art. As known in the art, there are a number of ways by which genes and gene constructs can be introduced into plants and a combination of transformation and tissue culture techniques have been successfully integrated into effective strategies for creating transgenic plants. For example, one skilled in the art will certainly be aware that, in addition to Agrobacterium-mediated transformation of Arabidopsis by vacuum infiltration (Bechtold 1993) or wound inoculation (Katavic 1994), it is equally possible to transform other plant species, using Agrobacterium Ti-plasmid mediated transformation (e.g., hypocotyl (DeBlock 1989) or cotyledonary petiole (Moloney 1989) wound infection), particle bombardment/biolistic methods (Sanford 1987; Nehra 1994; Becker 1994) or polyethylene glycol-assisted, protoplast transformation (Rhodes 1988; Shimamoto 1989) methods.

As will also be apparent to persons skilled in the art, and as described elsewhere (Meyer 1995; Datla 1997), it is possible to utilize plant promoters to direct any intended regulation of transgene expression using constitutive promoters (e.g., those based on CaMV35S), or by using promoters which can target gene expression to particular cells, tissues (e.g., napin promoter for expression of transgenes in developing seed cotyledons), organs (e.g., roots), to a particular developmental stage, or in response to a particular external stimulus (e.g., heat shock). Promoters for use herein may be inducible, constitutive, or tissue-specific or cell specific or have various combinations of such characteristics. Useful promoters include, but are not limited to constitutive promoters such as carnation etched ring virus (CERV), cauliflower mosaic virus (CaMV) 35S promoter, or more particularly the double enhanced cauliflower mosaic virus promoter, comprising two CaMV 35S promoters in tandem (referred to as a “Double 35S” promoter). Meristem specific promoters include, for example, STM, BP, WUS, CLV gene promoters. Seed specific promoters include, for example, the napin promoter. Other cell and tissue specific promoters are well known in the art.

Promoter and termination regulatory regions that will be functional in the host plant cell may be heterologous (that is, not naturally occurring) or homologous (derived from the plant host species) to the plant cell and the gene. Suitable promoters which may be used are described above. The termination regulatory region may be derived from the 3′ region of the gene from which the promoter was obtained or from another gene. Suitable termination regions which may be used are well known in the art and include Agrobacterium tumefaciens nopaline synthase terminator (Tnos), A. tumefaciens mannopine synthase terminator (Tmas) and the CaMV 35S terminator (T35S). Particularly preferred termination regions for use herein include the pea ribulose bisphosphate carboxylase small subunit termination region (TrbcS) or the Tnos termination region. Such gene constructs may suitably be screened for activity by transformation into a host plant via Agrobacterium and screening for the desired activity using known techniques.

Preferably, a nucleic acid molecule construct for use herein is comprised within a vector, most suitably an expression vector adapted for expression in an appropriate plant cell. It will be appreciated that any vector which is capable of producing a plant comprising the introduced nucleic acid sequence will be sufficient. Suitable vectors are well known to those skilled in the art and are described in general technical references. Particularly suitable vectors include the Ti plasmid vectors. After transformation of the plant cells or plant, those plant cells or plants into which the desired nucleic acid molecule has been incorporated may be selected by such methods as antibiotic resistance, herbicide resistance, tolerance to amino-acid analogues or using phenotypic markers. Various assays may be used to determine whether the plant cell shows an increase in gene expression, for example, Northern blotting or quantitative reverse transcriptase PCR (RT-PCR). Whole transgenic plants may be regenerated from the transformed cell by conventional methods. Such plants produce seeds containing the genes for the introduced trait and can be grown to produce plants that will produce the selected phenotype.

### Silencing

Silencing may be accomplished in a number of ways generally known in the art, for example, RNA interference (RNAi) techniques, artificial microRNA techniques, virus-induced gene silencing (VIGS) techniques, antisense techniques, sense co-suppression techniques and targeted mutagenesis techniques.

RNAi techniques involve stable transformation using RNA interference (RNAi) plasmid constructs (Helliwell 2005). Such plasmids are composed of a fragment of the target gene to be silenced in an inverted repeat structure. The inverted repeats are separated by a spacer, often an intron. The RNAi construct driven by a suitable promoter, for example, the Cauliflower mosaic virus (CaMV) 35S promoter, is integrated into the plant genome and subsequent transcription of the transgene leads to an RNA molecule that folds back on itself to form a double-stranded hairpin RNA. This double-stranded RNA structure is recognized by the plant and cut into small RNAs (about 21 nucleotides long) called small interfering RNAs (siRNAs). siRNAs associate with a protein complex (RISC) which goes on to direct degradation of the mRNA for the target gene.

Artificial microRNA (amiRNA) techniques exploit the microRNA (mlRNA) pathway that functions to silence endogenous genes in plants and other eukaryotes (Schwab 2006; Alvarez 2006). In this method, 21 nucleotide long fragments of the gene to be silenced are introduced into a pre-miRNA gene to form a pre-amiRNA construct. The pre-miRNA construct is transferred into the plant genome using transformation methods apparent to one skilled in the art. After transcription of the pre-amiRNA, processing yields amiRNAs that target genes which share nucleotide identity with the 21 nucleotide amiRNA sequence.

In RNAi silencing techniques, two factors can influence the choice of length of the fragment. The shorter the fragment the less frequently effective silencing will be achieved, but very long hairpins increase the chance of recombination in bacterial host strains. The effectiveness of silencing also appears to be gene dependent and could reflect accessibility of target mRNA or the relative abundances of the target mRNA and the hpRNA in cells in which the gene is active. A fragment length of between 100 and 800 bp, preferably between 300 and 600 bp, is generally suitable to maximize the efficiency of silencing obtained. The other consideration is the part of the gene to be targeted. 5′ UTR, coding region, and 3′ UTR fragments can be used with equally good results. As the mechanism of silencing depends on sequence homology there is potential for cross-silencing of related mRNA sequences. Where this is not desirable a region with low sequence similarity to other sequences, such as a 5′ or 3′ UTR, should be chosen. The rule for avoiding cross-homology silencing appears to be to use sequences that do not have blocks of sequence identity of over 20 bases between the construct and the non-target gene sequences. Many of these same principles apply to selection of target regions for designing amiRNAs.

Virus-induced gene silencing (VIGS) techniques are a variation of RNAi techniques that exploits the endogenous antiviral defenses of plants. Infection of plants with recombinant VIGS viruses containing fragments of host DNA leads to post-transcriptional gene silencing for the target gene. In one embodiment, a tobacco rattle virus (TRV) based VIGS system can be used.

Antisense techniques involve introducing into a plant an antisense oligonucleotide that will bind to the messenger RNA (mRNA) produced by the gene of interest. The “antisense” oligonucleotide has a base sequence complementary to the gene's messenger RNA (mRNA), which is called the “sense” sequence. Activity of the sense segment of the mRNA is blocked by the anti-sense mRNA segment, thereby effectively inactivating gene expression. Application of antisense to gene silencing in plants is described in more detail by Stam 2000.

Sense co-suppression techniques involve introducing a highly expressed sense transgene into a plant resulting in reduced expression of both the transgene and the endogenous gene (Depicker 1997). The effect depends on sequence identity between transgene and endogenous gene.

Targeted mutagenesis techniques, for example TILLING (Targeting Induced Local Lesions IN Genomes) and “delete-a-gene” using fast-neutron bombardment, may be used to knockout gene function in a plant (Henikoff 2004; Li 2001). TILLING involves treating seeds or individual cells with a mutagen to cause point mutations that are then discovered in genes of interest using a sensitive method for single-nucleotide mutation detection. Detection of desired mutations (e.g. mutations resulting in the inactivation of the gene product of interest) may be accomplished, for example, by PCR methods. For example, oligonucleotide primers derived from the gene of interest may be prepared and PCR may be used to amplify regions of the gene of interest from plants in the mutagenized population. Amplified mutant genes may be annealed to wild-type genes to find mismatches between the mutant genes and wild-type genes. Detected differences may be traced back to the plants which had the mutant gene thereby revealing which mutagenized plants will have the desired expression (e.g. silencing of the gene of interest). These plants may then be selectively bred to produce a population having the desired expression. TILLING can provide an allelic series that includes missense and knockout mutations, which exhibit reduced expression of the targeted gene. TILLING is touted as a possible approach to gene knockout that does not involve introduction of transgenes, and therefore may be more acceptable to consumers. Fast-neutron bombardment induces mutations, i.e. deletions, in plant genomes that can also be detected using PCR in a manner similar to TILLING.

Silencing of genes that encode cyclopeptide precursors may be useful to reduce levels of undesirable cyclopeptides in plants, and to facilitate production of a single cyclopeptide so as to simplify extraction/purification.

### EXAMPLE 1

### Identification of S. vaccaria Genes that Encode Putative Segetalin Precursors S. vaccaria RNA isolation and cDNA library construction

For cDNA library construction, total RNA was prepared from developing seed of S. vaccaria ‘Pink Beauty’ approximately 2-4 weeks after flowering. The polyA+RNA fraction was isolated (PolyATtract mRNA Isolation System, Promega) and used for cDNA library preparation with a SMART cDNA library construction kit (Clontech) according to the manufacturer's instructions using the vector pDNR-LIB. The cDNA library was called SVAR04NG.

### DNA Sequencing and Expressed Sequence Tag Analysis

Single bacterial colonies of the S. vaccaria cDNA library were inoculated in 96-well microtiter plates containing 150 μl aliquots of LB freezing medium (36 mM K₂HPO₄, 13.2 mM KH₂PO₄, 1.7 mM sodium citrate, 0.4 mM MgSO₄·7H₂O, 6.8 mM (NH4)₂SO₄, 4.4% (v/v) glycerol, 1% Bacto tryptone, 0.5% yeast extract, 0.5% NaCl) and kanamycin (50 μg/ml). After a 20 h incubation at 37° C. with shaking at 250 rpm, cells were either used immediately for the next step or stored at −80° C. DNA sequencing templates were prepared from 1 μl of the bacterial cell culture using the TempliPhi DNA Sequencing Template Amplification Kit (Amersham Biosciences, Piscataway, N.J.) according to the protocol provided by the manufacturer. The amplified products (1 μl) were used directly in a 20 μl cycle sequencing reaction. Sequencing was performed on an ABI3700 DNA sequencer using BigDye Terminator Cycle Sequencing Kit (Applied Biosystems, Foster City, Calif.) and the M13 reverse primer.

DNA sequencer traces were interpreted and vector and low quality sequences were eliminated using PHRED (Ewing 1998) and LUCY (Chou 2001). STACKPACK (Miller 1999) was used for clustering the resulting EST dataset. BLAST (Altschul 1990) was used to perform similarity searches.

The presence of numerous cDNA sequences showing a high degree of similarity, but appearing to encode different segetalin precursors required the use of special clustering parameters. The ESTs were translated in all 6 reading frames and then searched for exact matches to all circular permutations of known segetalin amino acid sequences. Each set of ESTs containing sequence that corresponded to (a single circular permutation of) a given segetalin amino acid sequence was clustered with CAP3 (Huang 1999) using the parameters minimum percent identity (p)=97 and overlap cutoff (o)=50.

### Identification of Saponaria ESTs Corresponding to Cyclopeptide Sequences.

A S. vaccaria developing seed expressed sequence tag collection developed previously (Meesapyodsuk 2007) was investigated for sequence relating to segetalin biosynthesis. Initially, six reading frame translations of the S. vaccaria EST database were searched for exact matches to all circular permutations of segetalin amino acid sequences. The presence of numerous cDNA sequences appearing to encode different segetalin precursors showing a high degree of similarity, required reclustering using special parameters. Each set of ESTs containing sequence that corresponded to a single circular permutation of a given segetalin amino acid sequence was first collected and then separately clustered with CAP3 (Huang 1999) using a minimum percent identity (p) of 97 and an overlap cutoff (o) of 50. To check the EST database for precursors of previously unknown segetalins, a TBLASTN search was conducted using the consensus amino acid sequence for the precursor of presegetalin A.

Analysis of S. vaccaria ESTs revealed nucleotide sequences encoding short 30-40 amino acid peptides which included the sequence of known segetalins. The ESTs in this group are highly abundant and comprise 14% of the total developing seed EST collection. The corresponding peptide sequences showed highly conserved N- and C-terminal domains which flanked the mature cyclic peptide sequences. These data are highly suggestive that cyclic peptides in S. vaccaria are biosynthesized ribosomally as linear precursors (presegetalins) which are then processed to mature cyclic peptides. Thus, it would appear that segetalin A is formed from (at least one) presegetalin A peptide encoded by a presegetalin A gene.

For clustering, putative presegetalin genes were first collected based on the presence of nucleotide sequences encoding mature cyclic peptide sequences. Added to this collection was an additional group of sequences which showed a high degree of similarity to members of the above collection. The collection was clustered with parameters which favored the clustering of sequences encoding the same mature cyclic peptide sequences, but not sequences encoding other CP sequences. Due to the large numbers of sequences involved, singletons were ignored in the sequence analysis. In general, more than one cluster was obtained for each segetalin. For example, for segetalin D, six clusters were found to have distinct cDNA sequences, which encode three distinct amino acid sequences, all of which include the same circular permutation of the mature segetalin D amino acid sequence. This gave rise to nomenclature in Table 4 using segetalin D as an example. sgd3b is a gene corresponding to the second of two cDNAs with distinct nucleotide sequences which encodes the third (preSGD3) of three putative segetalin D precursors. PreSGD3 is thought to give rise to segetalin D (SGD).

Interestingly, the sequence analysis also revealed cDNAs which a) showed predicted amino acid sequence similarity to the putative precursors of known segetalins and b) appeared to encode the precursors of novel segetalins. In the analysis of these predicted presegetalins only clusters containing more than 5 ESTs were considered (see

Table 5).

TABLE 4
Nomenclature for genes, precursors and mature cyclic peptides

| Entity | Long form | Short form |
|---|---|---|
| Gene | Presegetalin D3 gene b | sgd3b |
| Cyclic peptide precursor | Presegetalin D3 | preSGD3 |
| Cyclic peptide | Segetalin D | SGD |

TABLE 5
S. vaccaria genes encoding segetalin
precursors inferred from EST data

| | Segetalin | | |
|---|---|---|---|
| Segetalin | precursor | Gene | Contig Size |
| A | A1 | sga1a | 236 |
| | | sga1b | 2 |
| B | B1 | sgb1a | 133 |
| | | sgb1b | 2 |
| | B2 | sgb2 | 3 |
| D | D1 | sgd1 | 205 |
| | D2 | sgd2a | 191 |
| | | sgd2b | 2 |
| | | sgd2c | 2 |
| | D3 | sgd3a | 10 |
| | | sgd3b | 9 |
| F | F1 | sgf1a | 30 |
| | | sgf1b | 17 |
| | | sgf1c | 5 |
| | | sgf1d | 3 |
| G | G1 | sgg1 | 33 |
| H | H1 | sgh1 | 128 |
| | H2 | sgh2 | 4 |
| GRVKA | GRVKA1 | grvka1 | 30 |
| GLPGWP | GLPGWP1 | glpgwp1 | 7 |
| FGTHGLPAP | FGTHGLPAP1 | fghglpap1 | 28 |

Based on the sequence analysis, there appear to be at least 21 S. vaccaria genes (or alleles) encoding 13 (precursor) amino acid sequences, which include the sequences of six known segetalins and three putative segetalins. The known segetalins represented are A, B, D, F, G and H. This matches well with the segetalins which have been detected chemically in the Pink Beauty variety (A,B,D,F,G,H; Table 5). In comparison with the precursor sequences of the known segetalins, the unknown segetalins are predicted to be different by having the sequences GRVKA, GLPGWP or FGTHGLPAP (see FIG. 1).

### EXAMPLE 2

### Demonstration that Segetalins Are Produced Ribosomally

To test the possibility that S. vaccaria cyclic peptides are produced from ribosomally-produced precursors, hairy root cultures were generated which express presegetalin A1. The variety White Beauty was used, since it was found not to produce segetalin A (Table 5).

Preparation of the Over-Expression Plasmid Containing sga1a

Plasmid DNA was prepared from the Saponaria vaccaria ‘Pink Beauty’ developing seed EST library (Meesapyodsuk 2007) clone, SVARO4NG_{—}04E02 using the QIAprep mini spin kit (QIAGEN). The preSGA1 ORF was amplified using Vent DNA polymerase (New England Biolabs) and the primers, JC1 (5′-CACCATGTCTCCAATCCTC-3′-SEQ ID NO: 52) and JC2 (5′-TTACACAGGGGCTGAAGC-3′-SEQ ID NO: 53). The 103-bp PCR product was gel-purified using QIAEXII (QIAGEN) and cloned into the Gateway entry vector pENTRID-TOPO (Invitrogen). The DNA sequence was verified using the Big Dye terminator cycle sequencing kit (Applied Biosystems Inc.) with an ABI3700 DNA sequencer. LR Clonase II (Invitrogen) was used to transfer the insert into the binary over-expression plant transformation vector pK7WG2D (Karimi 2002). After DNA sequence verification, the resultant plasmid, pJC003, was used to transformed electrocompetent cells of Agrobacterium rhizogenes LBA9402. A. rhizogenes LBA9402 was also transformed with pK7WG2D alone. PCR was used to confirm transformation (see below).

Transformation of S. vaccaria

Sterile leaf explants of S. vaccaria ‘White Beauty’ (which does not contain segetalin A—see Table 3) were transformed separately with either pJC003 or pK7WG2D and hairy roots were regenerated as described previously (Schmidt 2007). Rapidly growing lines that showed kanamycin resistance and GFP fluorescence with no bacterial contamination were used to establish single hairy root lines. All transgenic hairy root lines originated from independent GFP-positive adventitious roots.

### Hairy Root DNA Extraction and PCR Analysis

DNA was extracted from a 100-200 mg sample of each root culture using the DNeasy Plant Mini Kit (Qiagen) and subjected to multiplex PCR analysis to simultaneously score for the presence or absence of the rolC, virD, egfp and nptII genes as described previously (Schmidt 2007). To confirm that kanamycin-resistant and egfp-positive hairy roots were transformed, the presence of the sga1a gene was verified by PCR. The PCR reaction mixture (25 μl) contained 1 μl of DNA, as prepared above, in 1×PCR reaction buffer, 2.5 mM MgCl₂, 0.2 mM of each dNTP, 0.4 μM of each primer (JC3 5′-CCGACAGTGGTCCCAAAGATG-3′ (vector-specific) (SEQ ID NO: 54) and JC4 5′GCCTGAAAAGCCCAAACTGG-3′ (gene-specific) (SEQ ID NO: 55)) and 5 U Taq DNA polymerase (Invitrogen). Amplification was performed in a Stratagene Robocycler Gradient 96 using the following program: 94° C. for 10 min, 30 cycles of 94° C. for 30 s, 62° C. for 40 s, and 72° C. for 50 s, followed by 72° C. for 10 min. The expected size of the PCR fragment was 398 bp.

### Hairy Root Sample Preparation for LC/MS

For each transformed hairy root line, 1.2-2.2 g fresh weight of hairy roots were added to 5 ml methanol in a 10 ml glass screw-top tube and homogenized using a Polytron (Kinematica, Bohemia, USA). The sample was sonicated for 20 min using a Branson 2510 ultrasonic cleaner (Branson Ultrasonic Corporation, Danbury Conn.), centrifuged at 1,400×g for 3 min and the supernatant was transferred to a new tube. An additional 5 ml methanol was added to the pellet and sonicated, centrifuged and decanted, as above. This step was repeated once more. A tube containing the combined supernatants was placed in a heating block at 30-35° C. and the methanol was evaporated under a nitrogen stream. The sample was resuspended in 1 ml distilled H₂O, transferred to a 1.5 mL tube, and centrifuged at 12,000×g for 5 min. The supernatant was then placed in a Costar SPIN-X® (0.22 μm cellulose acetate; Corning, Corning, USA) centrifuge filter unit and centrifuged at 12,000×g for 1 min. The filtrate was then used for analysis by LC/MS.

### Liquid Chromatography/Mass Spectrometry (LC/MS)

A 2695 Alliance chromatography system, with inline degasser, coupled to a ZQ mass detector and a 2996 photodiode array detector (Waters, Milford Mass.) was used for LC-MS-PDA analysis. MassLynx software was used for data acquisition and analysis. The column used was a Waters Sunfire 3.5-μm RP C-18 150×2.1 mm. The flow rate was 0.15 ml/min. The column was maintained at 35° C. during analysis. The binary solvent system consisted of 90:10 v/v water/acetonitrile containing 0.12% acetic acid (solvent A) and acetonitrile containing 0.12% acetic acid (solvent B). The gradient program used was 0-8 min, 95: 5 A/B; 8-31 min, 95:5 to 50:50 A/B; 31-33 min, 50:50 to 0:100 A/B; 33-48 min, 0:100 A/B. Voltage parameters for negative electrospray ionization (ESI-) were: capillary, 2.80 kV; cone, ramped from −15 to −45 V; extractor, −3.00 V; RF lens, −0.5 V; for positive electrospray ionization (ESI⁺), they were: capillary, 3.50 kV; cone, ramped from +15 to +45 V; extractor, 6.00V; RF lens, 0.9 V.

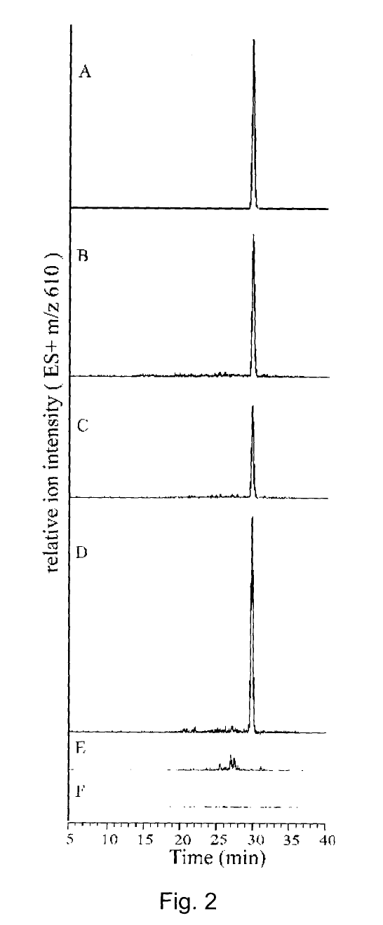

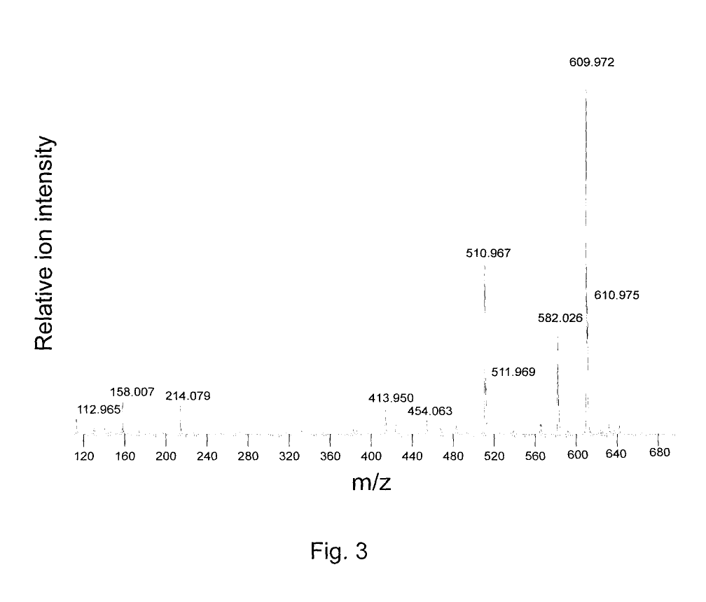

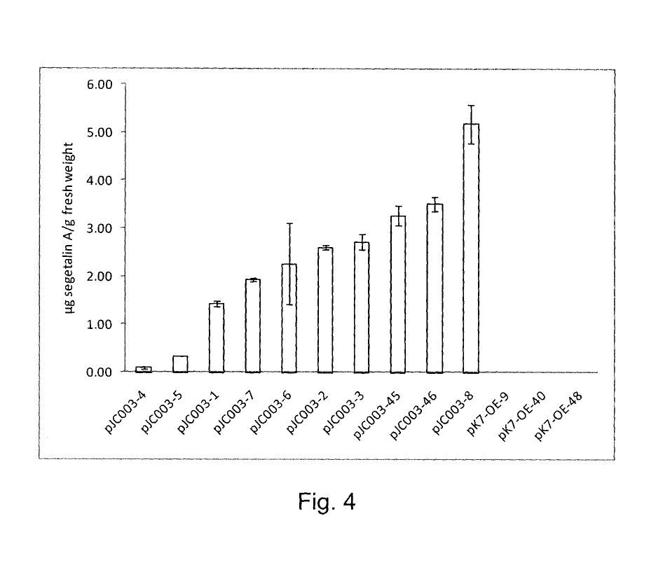

FIG. 2 shows the results of LC/MS analysis of hairy root samples. Hairy root lines which were not engineered to express presegetalin A did not contain detectable amounts of segetalin A (FIG. 2B, 2E, 2F). On the other hand, independent hairy root lines expressing presetalin A were found to contain segetalin A in the range of 0.1-5 μg/g fresh weight, based on coelution of a compound with segetalin A and which gave rise to a fragment ion of m/z=610 (FIGS. 2B and 2C).

### EXAMPLE 3

### Methods for Recovery of Segetalin Cyclopeptides from Saponaria vaccaria

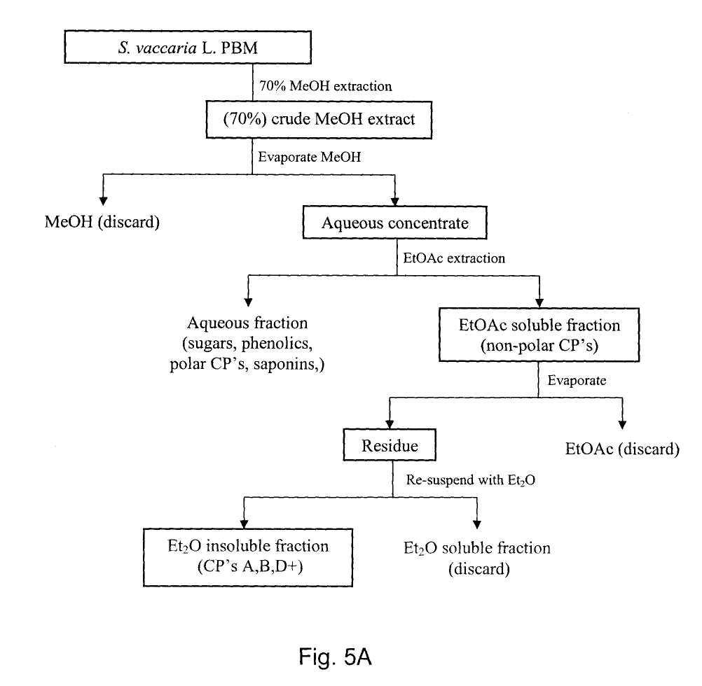

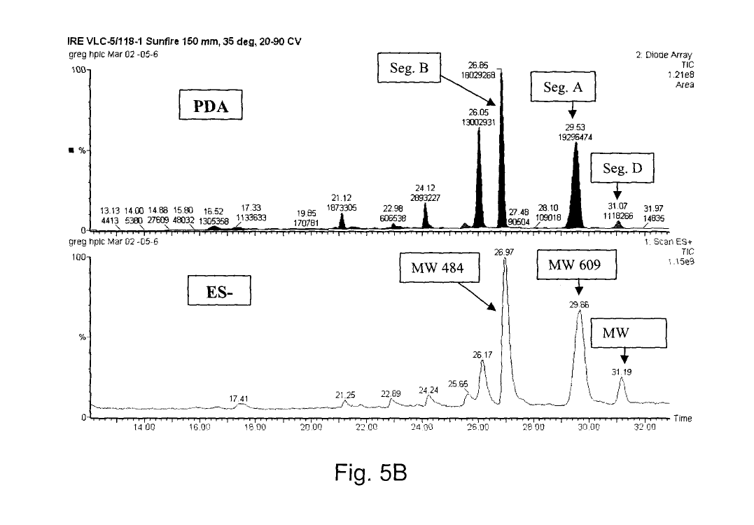

Three known cyclopeptides (segetalin A, B and D) were purified from PC seed extracts. A cyclopeptide containing fraction ‘CP's A,B,D+’ was obtained from the 70% MeOH extract of the seed as follows: an aqueous concentrate of the dry MeOH extract was extracted with ethyl acetate (EtOAc, 2x) and the EtOAc soluble fraction separated and evaporated to dryness. The dry residue was then re-suspended in diethyl ether (Et₂O) to eliminate non-polar impurities, and the Et₂O insoluble fraction was labeled as ‘CP's A,B,D+’. A diagram of the extraction procedure is shown below (FIG. 5A).

Cyclopeptides (CP's) were then purified from the Et₂O insoluble fraction ‘CP's A,B,D+’ by vacuum liquid chromatography (VLC). Cyclopeptide mixture (5 g) was loaded dry on top of the column, and a gradient of a mixture of EtOAc: acetic acid/water (1:1) was passed through collecting 100 mL fractions. Gradient concentrations were from 12:1, with a decrease in the concentration of EtOAc by 4.16% for each fraction. The final concentration used was 5:1. Fifteen 100 mL fractions were collected, aliquots were analysed by LC-MS-DAD, and crystallized pure cyclopeptides segetalin A and B, 80% pure segetalin D was purified by consecutive preparative thin layer chromatography (PTLC) using a mixture of EtOAc:acetic acid:water (9:0.5:0.5). A chromatogram from an impure mixture of the cyclopeptides is shown below (FIG. 5B).

### EXAMPLE 4

### Obtaining Segetalin A from a Cyclopeptide-Enriched Fraction

### Extraction

The germ extract from Saponaria vaccaria was dissolved in distilled water and heated to approximately 50° C. with constant stirring. The non-polar fraction (enriched with non-polar cyclopeptides) was extracted using ethyl acetate. A second and third extraction on the aqueous phase was performed to ensure maximum removal of the non-polar compounds. The organic fraction was concentrated via rotor-evaporation and defatted using diethyl ether. Vacuum filtration was conducted to recover the cyclopeptides (residue) from the fats. The diethyl ether (Et₂O) insoluble fraction was analyzed by HPLC-PDA-MS. The chromatogram showed three main peaks corresponding to Segetalin B (Rt 27.20 min), Segetalin A (Rt 29.92 min) and Segetalin D (Rt 31.48 min).

An alternative method for obtaining a cyclopeptide-enriched fraction was developed by a 95% ethanol precipitation on the germ extract. The aqueous germ extract was dried and resuspended in 95% ethanol (solid to solvent ratio of 1:20) and stirred for approximately 1 h, then filtered to remove the precipitates formed. HPLC-PDA-MS analyses indicated that the non-polar cyclopeptides Segatalin A, B, and D were predominantly in the filtrates. The filtrate was evaporated to dryness and then resuspended in distilled water. The cyclopeptides were extracted with ethyl acetate followed by a defatting step as previously described.

### Cyclopeptide Fractionation

The defatted organic phase was ground and resuspended in ethyl acetate/50% acetic acid (12:1). The sample was sonicated prior to application on a 5 cm column of TLC grade Si-gel (internal diameter 6.8 cm). Vacuum liquid chromatography (VLC) was conducted using a solvent system of ethyl acetate/50% acetic acid (12:1). A gradient was applied until the ratio of ethyl acetate to 50% acetic acid was 5:1. Following each elution, fractions were concentrated in vacuo and set in a 70° C. water bath.

### Isolation of Segetalins

After evaporation to dryness, fractions containing mainly segetalin A and B were combined. A minimum volume of absolute ethanol was added and the sample heated until partial solubility was attained. The residue was removed via gravity filtration and rinsed in ethanol to ensure complete removal of the entrained solution. The remaining mother liquor was heated until completely dissolved and stored at room temperature. After about 24 h, a white precipitate was observed. This precipitate was extracted via centrifugation and rinsed with cold ethanol. Based on HPLC-PDA-MS analyses, the first residue and second precipitate were segetalins B and A, respectively. Successive crystallizations using ethanol were conducted on the same sample until the mother liquor yielded negligible crops of segatalin A.

### Purification

Samples were resuspended in a solution of acetonitrile with 0.01% acetic acid prior to loading onto a 20 cm×20 cm PTLC 1000 μm plate. The eluting solvent was a mixture of ethyl acetate, acetic acid and distilled water in the ratio 9:0.5:0.5. The plate was run four times using UV visualization after each run. The fluorescent region observed (Rf about equal to 0.5 or 0.6) was scraped off and resuspended in acetonitrile with 0.01% acetic acid (50 mL). Samples were stirred for about 15 min followed by vacuum filtration. Filtrates were analyzed via HPLC-PDA-MS and displayed purity of the segetalin of interest.

### EXAMPLE 5

### Cyclolinopeptide Gene Characterization in Flax

Construction of Flax Seed cDNA Libraries

Total RNAs were isolated independently from flax (Linum usitatissimum cultivar Bethune) seed tissues representing five embryo developmental stages (globular, heart, torpedo, cotyledonary and mature), two seed coat stages and one pooled endosperm tissues and corresponding cDNA libraries were constructed. The libraries contain about 1.5 kb average cDNA inserts. These flax seed cDNA libraries were used to generate about 150,000 ESTs by sequencing from the 3′ end of the inserts It was anticipated that because significant amounts of several cyclopeptides are found in flax seeds, that these are derived from precursor proteins encoded by gene(s) expressed in flax seeds.

In order to search for sequences related to cyclic peptide production, the flax ESTs were translated in all six reading frames. A computer search on the resulting amino acid sequences was the made with all circular permutations of the known flax cyclic peptides. This led to the detection of over 200 ESTs that appear to correspond to a single gene called CP1, encoding a precursor to three cyclic peptides. The majority of these ESTs were identified from the cotyledonary stage embryo cDNA library suggesting the expression of the corresponding gene is developmentally regulated. The cDNA clones (CP1) with the full predicted coding sequence (from the start to stop codons) have been identified and the sequence details are shown in SEQ ID NO: 33 and SEQ ID NO: 34.

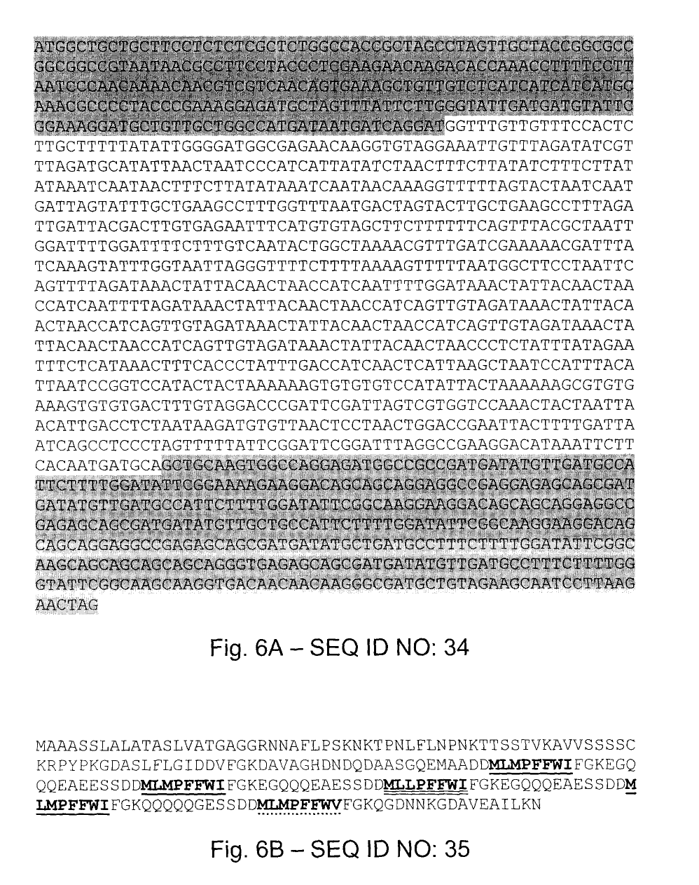

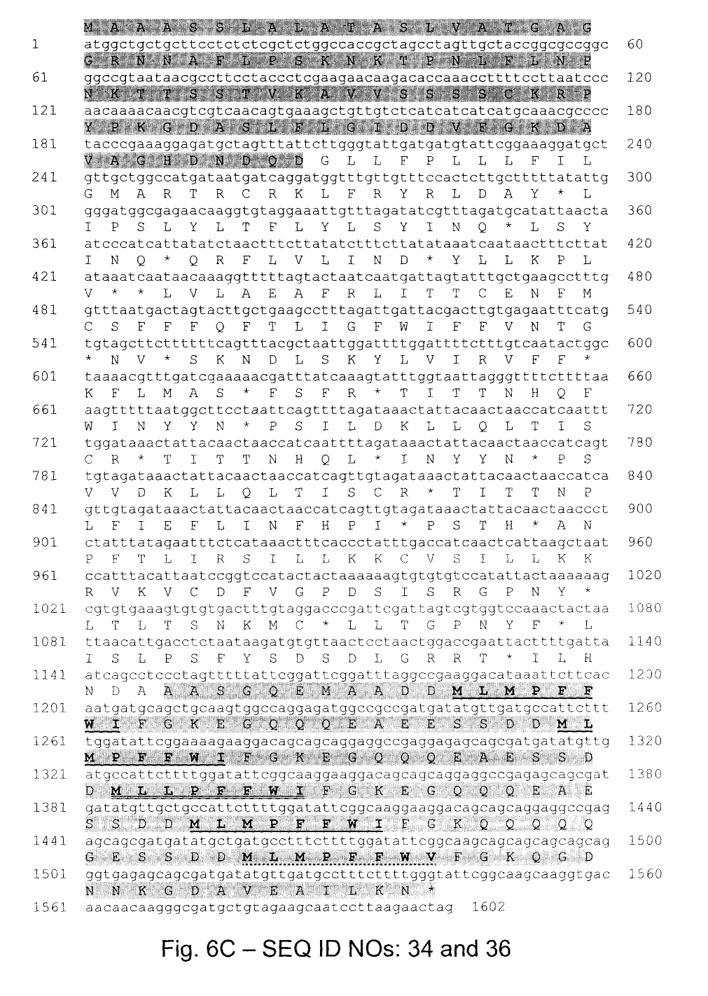

The analysis of cDNA sequences suggests that these are likely expressed from the same gene. To identify the corresponding genomic sequence, primers at the 5′ and 3′ ends of the cDNA clones were designed and PCR reaction performed using the flax genomic DNA. This reaction produced one band corresponding to an about 1600 by fragment that was cloned into vector pCR2.1 (Invitrogen). Complete nucleotide sequence of this DNA fragment was determined and the analysis revealed a perfect match with the cDNA sequence and the presence of a single intron (942 bp) representing the CP1 genomic clone (sequence details presented in FIG. 6). Analysis of this sequence showed that all the five cyclopeptide encoding sequences are present in the second exon. The CP1 encoded protein contains three copies of eight amino acid cyclopeptide with “MLMPFFWI” (SEQ ID NO: 37) composition. Additionally single amino acid variants resulting in cyclopeptides containing “MLLPFFWI” (SEQ ID NO: 38) and “MLMPFFWV” (SEQ ID NO: 39) are represented by one copy of each. All five putative cyclopeptide sequences are flanked by conserved a “DD” at the 5′ end and “FGK” at the 3′ end suggesting an important functions for these sequences in the processing and release of peptides from the precursor protein. The analysis also identified the presence of two putative chloroplast targeting signals in the CP1 protein, including an N-terminal signal peptide. The implication of this finding is that it is possible that the nuclear encoded gene product(s) is/are targeted to chloroplast for further processing. The putative targeting of the CP1 precursor protein to the chloroplast raises the possibility that the chloroplast genome may carry additional gene sequences corresponding to the additional cyclopeptides known from flax seed.

### EXAMPLE 6

### Cyclolinopeptide Gene Expression in E. coli

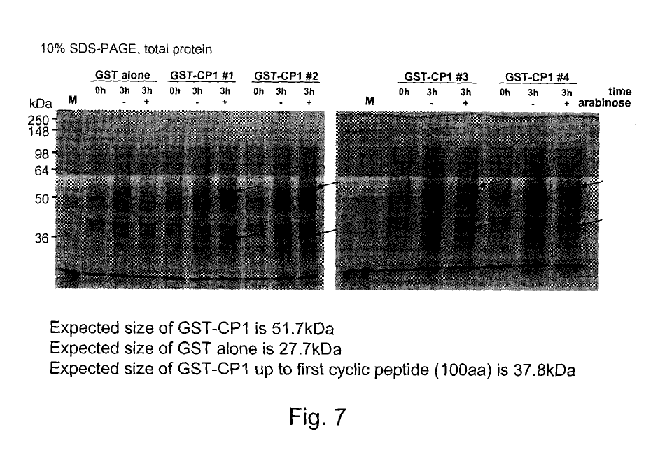

To further characterize the isolated flax CPI cDNA, an inducible recombinant GST-CP1 construct was prepared and introduced into E. coli. An induced protein with a molecular weight similar to that predicted for the GST-CP1 fusion protein (51.7 kDa) was observed. Additionally, a smaller prominent band was also observed under induction conditions. The size of this protein was similar to the predicted 37.8 kDa size of GST+(CP1 precursor protein minus the predicted cyclopetides) suggesting cleavage and/or processing at the 5′ end of the first cyclopetide sequence. This observation raises the possibility that the CP1 precursor protein contains the necessary structural and/or processing signals recognized in the heterologous prokaryotic E. coli system. The details of SDS-PAGE analysis is presented in FIG. 7.

### EXAMPLE 7

### Flax CP1 Overexpression in Transgenic Flax Seeds

### Plant Transformation Construct:

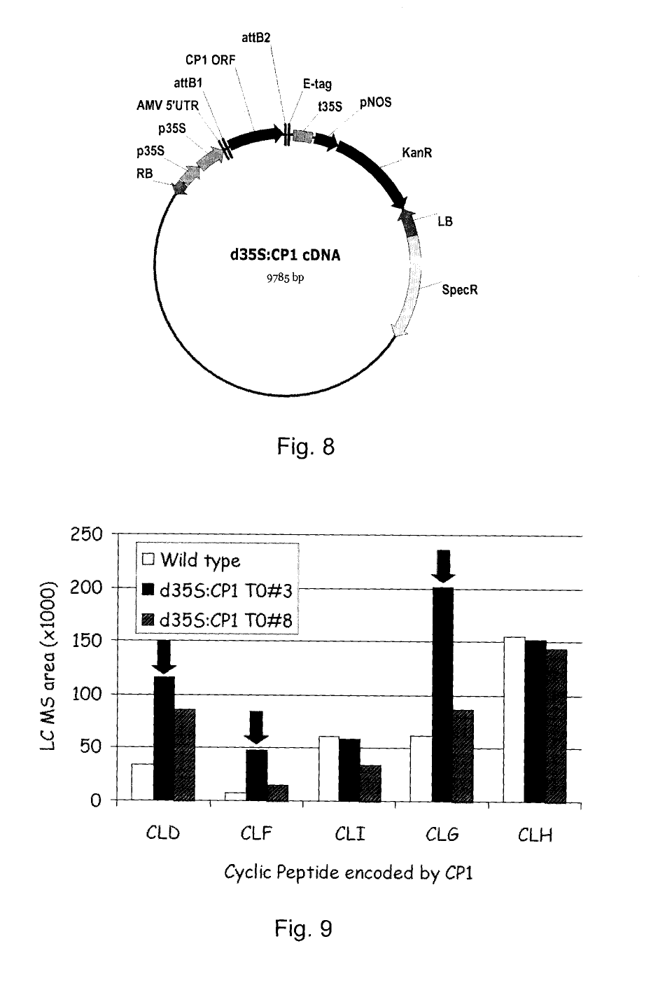

CP1 ORF was amplified by per from a full-length EST identified from a Flax CDC Bethune Cotyledon staged embryo library using primers CP1-F (5′-GCGGCCGCATGGCTGCTGCTTCCTCTCTCGCT-3′-SEQ ID NO: 56) and CP1-R1 (5′-CCTGCAGGCTAGTTCTTAAGGATTGCTTCTACAGCATC-3′-SEQ ID NO: 57). This resulted in the addition of NotI and SbfI restriction enzyme sites added immediately 5′ to the start codon and 3′ to the stop codon, respectively. This amplicon was TA cloned into pCR2.1 (Invitrogen) to create CP1 cDNA pCR2.1. The GATEWAY entry vector pER380 NSX was created by NotI AscI digestion of an insert containing pENTR/D-TOPOR (Invitrogen) to remove the insert, followed by ligation with a NotI AscI digested synthesized linker (5′-GCGGCCGCAAAAAACCTGCAGGACCCGGGAGGCGCGCC-3′-SEQ ID NO: 58) in order to add SbfI and XbaI restriction sites between NotI and AscI in the multicloning site. CP1 cDNA pCR2.1 and pER380 NSX were both NotI SbfI double digested and resulting fragments were separated with an agarose gel. The CP1 cDNA insert and pER380 NSX backbone fragments were excised, gel eluted and ligated together with T4 DNA ligase to create entry vector CP1 cDNA pER380 NSX. Gateway Agrobacterium tumefaciens destination vector pER330 (Teerawanichpan 2007) was modified by the addition of a second 35SCaMV promoter and 5′UTR of AMV, resulting in pER370. LR Clonase II (Invitrogen) reaction was performed with CP1 cDNA pER380 NSX and pER370 to make d35S:CP1 cDNA expression vector (FIG. 8). d35S:CP1 cDNA was transformed into Agrobacterium GV3101::pMP90 through triparental mating.

### Flax Transformation Procedure:

Flax seeds (CDC Normandy) sterilized with 70% ethanol and 30% bleach, and rinsed with sterile distilled water. Seeds spread on dishes containing germination medium (½ strength MS minimal organics medium, 10 g/l sucrose, pH 5.8, 0.7% phytagar). Plates were sealed, covered with foil and placed at 24° C. for 4-5 days to germinate and become etiolated.

d35S:CP1 cDNA Agrobacterium LB cultures containing gentamycin (25 mg/l) and spectinomycin (100 mg/l) (2×50 ml) inoculated from smaller cultures and grown at 28° C. approximately 24 h. Each culture centrifuged at 5000 rpm for 10 minutes at room temperature to pellet Agrobacterium. Each pellet resuspended in 50 ml sterilized resuspension medium (MS salts basal medium, 30 g/l sucrose, 1 mg/l BAP, 0.02 mg/l NAA, pH 5.8). Each Agrobacterium resuspension was split in two to yield a total of four tubes of 25 ml resuspension cultures. A small spatula tip of sterile carborundum powder was added to some of the resuspension cultures to increase explant wounding potential.

Using aseptic technique, etiolated hypocotyls were cut into 2-5 mm pieces, added into a resuspension culture tube and vortexed 30 s. Culture containing explants was poured into a deep 100×25 mm petri dish to gently shake for 15-20 min. Agrobacterium resuspension was removed from the explants with a sterile transfer pipette and explants were transferred to a deep petri dish containing two sterile filter papers dampened with sterile resuspension medium. Sealed plates were covered with foil and left to co-cultivate at 22° C. for 6-7 days, rewetting filters with sterile resuspension medium after first 2-3 days.

Hypocotyl explants aseptically transferred to selection medium (MS salts basal medium, 30 g/l, 1 mg/l BAP, 0.02 mg/l NAA, pH 5.8, 0.7% phytagar, autoclaved and allowed to cool slightly before adding 600 mg/l Timentin and 200 mg/l kanamycin). 30-50 explants per deep dish. Plates put at 24° C. with a 16 h photoperiod.

After 2 weeks green callus develops at cut ends. First green shoots after approximately 3 weeks and continues to develop for several more weeks. Emerging shoots cut and placed in elongation/rooting medium (MS salts basal medium, 20 g/l sucrose, pH 5.8, 0.7% phytagar, autoclaved and allowed to cool slightly before adding 600 mg/l Timentin and 150 mg/l kanamycin). Shoots continuously harvested as they developed. Kanamycin resistant shoots will develop roots and will remain slightly greener than sensitive shoots in the presence of kanamycin. Confirmed seedlings were transgenic by pcr. Once good roots formed, transgenics were transferred to soil. Transgenic flax and wild type controls grown in growth cabinet (22° C. day/18° C. night, 16 h photoperiod). Seeds harvested after plants dry. Non-seed tissues removed from seeds.

Preparation of Flax seed extracts for LC MS Analysis:

d35S:CP1 cDNA Normandy T1 seeds from T0 plants #3 and #8 ground with mortar and pestle. Wild type Normandy seeds from plant growing alongside the transgenic plants were ground for a control. 120 mg ground seed weighed out and extracted with 1.2 ml 80% methanol by sonicating 15 minutes twice, vortexing in between. Ground seed suspensions were microfuged 5 minutes and 80% methanol-soluble supernatant was transferred to a fresh 2 ml microfuge tube and dried down under nitrogen. Added 300 μl 80% methanol to each tube, vortexing and sonicating to resuspend the concentrated 80% methanol extracts. The extract was filtered through 0.2 μm nylon filters (13 mm diameter) into a sample vial.

### HPLC-PAD-MS Analysis of 80% Methanol-Soluble Flax Seed Extracts:

HPLC-PAD-MS was performed on a Waters 2695 Alliance chromatography system with inline degasser, coupled to a ZQ2000 mass detector and a 2996 photodiode array detector. A Waters Sunfire column 3.5μ RP C₁₈ 150×2.1 mm was used and maintained at 35° C. during runs. MassLynx™ 4.0 software was used for data aquisition and manipulation. Methods were followed as outlined in Balsevich 2009 with the following modifications:

Gradient: solvent A, 0.1% acetic acid in 10% acetonitrile (aq. v/v) and solvent B, 0.1% acetic acid in 100% acetonitrile. A linear gradient of 65% A: 35% B at 0 min to 0% A: 100% B at 35 min was run at a flow rate of 0.2 ml/min.

ZQ temperatures: source (° C.) 120 and desolvation (° C.) 320.

The mass detector parameters (ES+) were set to: capillary (kV) 2.8, scan (m/z) 850-1150 with cone voltage ramp (V) 45-60, extractor (V) +3 and RF lens (V) +0.5. The diode array detection was performed at 200-400 nm.

Sample injection quantity (μl) 25.

MassLynx™ 4.0 software used to calculate integration of areas under peaks of CP1 cDNA encoded cyclic peptides (MW): CLD (1064), CLF (1084), CLG (1098), CLH (1082) and CLI (1068).

### Results:

Some of the flax cyclic peptides biochemically isolated and reported in the literature have post-translational amino acid modifications, not encoded in the DNA sequence. Table 6 shows the cyclic peptide sequences encoded by CP1, their SEQ ID NO:, and their biochemically isolated counterparts. SEQ ID NO: 37 refers to CLG and CLH. SEQ ID NO: 38 refers to CLD. SEQ ID NO: 39 refers to both CLF and CLI. LC MS analysis of 80% methanol T1 seed extracts from two independent d35S:CP1 cDNA flax lines demonstrated that ectopic expression of CP1 cDNA in flax seeds leads to the increased levels of CLD, CLF and CLG (FIG. 9) which corresponds to one biochemical form from each of the three sequences, SEQ ID NOs: 37, 38 and 39.

TABLE 6
Comparison of Biochemically Isolated Cyclic Peptides
to Cyclic Peptides Derived from DNA Translation

| | |
|---|---|
| Biochemically Isolated | Translation from DNA |

| Name | Sequence | Sequence | SEQ ID NO: |
|---|---|---|---|
| CLD | PFFWIMsoLL | PFFWIMLL | 38 |
| CLF | PFFWVMsoLMso | PFFWVMLM | 39 |
| CLI | PFFWVMLMso | | |
| CLG | PFFWIMsoLMso | PFFWIMLM | 37 |
| CLH | PFFWIMsoLM | | |

Mso = methionine sulfoxide

### EXAMPLE 8

### Identification of Citrus Cyclic Peptide Precursor mRNA and Amino Acid Sequences

A number of cyclic peptides have been isolated and characterized from the genus Citrus (Morita 2007). This includes cyclic peptides with the sequence GLVPS (SEQ ID NO: 41) and GLLLPPFG (SEQ ID NO: 43). In order to identify nucleotide sequences encoding cyclic peptide precursors, Citrus expressed sequence tags collected in Genbank were translated in all six reading frames. A computer search was made for all circular permutations of GLVPS and GLLPPFG in the translated sequences. Included in the results were matches to Genbank accessions numbered DN798249 (corresponding to a Star Ruby grapefruit temperature-conditioned flavedo cDNA Citrus×paradise cDNA) and EG026628 (corresponding to a Citrus clementina cDNA). The amino acid sequences of the open reading frames which include the mature cyclic peptide sequences are shown in SEQ ID NOs: 40 and 42.

To one skilled in the art, one would normally consider matches to peptides of 6-8 amino acid of questionable value, since such matches would be considered statistically insignificant. However, there is a notable similarity between the two sequences in length and sequences near the mature cyclic peptide sequence and this suggests that the above matches are not random. Furthermore, it suggests that the corresponding messenger RNAs give rise to precursors with the amino acid sequence shown, which are subsequently processed to mature cyclic peptides with sequences GLVLPS and GLLLPPFG. Furthermore, if a TBLASTN search of expressed sequence tags in Genbank is performed using the amino acid shown for DN798249, numerous sequences are found to encode a similar amino acid sequence which appears to represent the precursor of a cyclic peptide with the sequence GYLLPPS (SEQ ID NO: 45) in Citrus sinensis. An example of this is the Genbank accession numbered DC900394 (corresponding to Citrus sinensis cDNA clone VS28967) with the predicted amino acid sequence as shown in SEQ ID NO: 44.

On this basis, one skilled in the art would predict a cyclic peptide with the sequence GYLLPPS, or a posttranslational modification thereof, which is derived from the precursor protein with the amino acid sequence shown, and ultimately the gene encoding the amino acid sequence. Indeed, GYLLPPS corresponds to cyclonatsudamine A, a vasodilator cyclic peptide from Citrus natsudaidai (Morita 2007).

### EXAMPLE 9

### Identification of Carnation Peptide Precursor mRNA and Amino Acid Sequences

A number of cyclic peptides have been isolated and characterized from other members of the Caryophyllaceae (Tan 2006)). In order to identify nucleotide sequences encoding cyclic peptide precursors related to those of Saponaria vaccaria, a TBLASTN search of expressed sequence tags in Genbank was performed using the amino acid of presegetalin A. Sequences were found to encode similar amino acid sequences including those corresponding to Genbank accessions numbered AW697819 (corresponding to carnation flower specific cDNA library Dianthus caryophyllus cDNA clone HM002), AW697902 (corresponding to carnation flower specific cDNA library Dianthus caryophyllus cDNA clone HM085) and CF259529 (corresponding to subtracted carnation petal cDNA library Dianthus caryophyllus cDNA clone Dc080). The corresponding amino acid sequences for these accessions are SEQ ID NO: 46, SEQ ID NO: 48 and SEQ ID NO: 50, respectively. Based on similarity to the S. vaccaria cyclic peptide precursor sequences, these appear to represent the precursors of carnation cyclic peptides, which include, but may not be limited to GPIPFYG (SEQ ID NO: 47), GLPYEQ (SEQ ID NO: 49) and GYKDCC (SEQ ID NO: 51).

Free List of Sequences:
SEQ ID NO: 1-sga1a-consensus cDNA (517 bp) encoding preSGA1 (S. vaccaria)
GACCGTTAACAATCTTGTAATTTAGTGTGTACAAGCTCTATAAATAGAGGCAAGTAATGT
GGCCATAAAAGGACACACAAAAAACATTCAAACAAATCATTTAATCTCTAACTTTACAAG
TCCAATACTTTATTTGTGAAAATGTCTCCAATCCTCGCCCACGACGTAGTCAAGCCCCAA
GGTGTCCCAGTTTGGGCTTTTCAGGCAAAAGATGTTGAAAATGCTTCAGCCCCTGTGTAA
ATTAATGTACACAATGCGCTTCTTCGGCCTTTAGATACGATGTTTCCAACCAAAATAAAC
CATAATGTTATGTCGAGTGTCATGTTTCTTATTTCTGTAATTTTATTTCTGTATATTGTT
TCGATTTTTAAATTGAAACAATAAACTATGTTAACTGGTTTGTAATAAAATCTAAAAGGC
CGTTCTAGTGTAAATTTAAGCATTCTCCTGTCGTTCATTTCTCCTTAGACACATTAAACC
ATACTAAGATAATATAATTTTGAACTCAAAATATTAT
SEQ ID NO: 2-preSGA1-linear polypeptide (32 aa) encoded by sga1a (S. vaccaria)
MSPILAHDVVKPQGVPVWAFQAKDVENASAPV
SEQ ID NO: 3-Segetalin A-cyclic polypeptide (6 aa) from preSGA1 cyclization (S.
vaccaria)
GVPVWA
SEQ ID NO: 4-sgb1a-consensus cDNA (445 bp) encoding preSGB1 (S. vaccaria)
GGGACAGTCGGGGACACACAAAAAACATTCAAACAAATCATTTAATCTCTAACTTTACAA
GTCCAATACTTTATTTGTGAAAATGTCTCCAATCCTCGCCCACGACGTAGTCAAGCCCCA
AGGTGTAGCTTGGGCTTTTCAGGCAAAAGATGTTGAAAATGCTTCAGCCCCTGTGTAAAT
TAATGTACACAATGCGCTTCTTCGGCCTTTAGATACGATGTTTCCAACCAAAATAAACCA
TAATGTTATGCCGAGTGTCATGTTTCTTATTTCTGTAATTTTATTTATGTATATTGTTTC
GATTTTTAAATTGAAACAATAAACTATGTTAATTGGTTTGTAATAAAATCTAAAGGCCGT
TCTAGCGTAAATTTAAGCATTCGCCTGTCGTTCATTTCTCCAAAGACATCATTAAACCAT
ACTAAGATAATATAATTTTGAACCC
SEQ ID NO: 5-preSGB1-linear polypeptide (31 aa) encoded by sgb1a (S. vaccaria)
MSPILAHDVVKPQGVAWAFQAKDVENASAPV
SEQ ID NO: 6-preSGB2-linear polypeptide (31 aa) (S. vaccaria)
MSPILAHDVVKPQGVAWAFQAKDAENASSPV
SEQ ID NO: 7-Segetalin B-cyclic polypeptide (5 aa) from preSGB1 or preSGB2
cyclization (S. vaccaria)
GVAWA
SEQ ID NO: 8-sgd1-consensus cDNA (365 bp) encoding preSGD1 (S. vaccaria)
GAATCACACACAAAATAAATTCATACAAATCATTTATTTAGTCTCTAACTTACAAACTCC
AATACTTCATTTGTGAAAATGTCTCCAATTTTTGCCCACGACGTAGTCAACCCCCAAGGC
CTAAGTTTCGCTTTTCCGGCAAAAGATGCTGAAAATGCTTCATCCCCGGTGTAAACTTAT
GTACACAATGCGCTTCTTCGGCCTTTAGATACGATGTTTCCAACCAAAATAAACCATAAT
GTTATGTCGAGTGTCATGTTTCTTATTTCTGTAATTTTATTTCTGTATATTGTTTCGATT
TTTAAATTGAAACAATAAACTATGTTAACTGGTTTGTAATAAAATCTAAAAGGCCGTTCT
AGTAC
SEQ ID NO: 9-preSGD1-linear polypeptide (31 aa) encoded by sgd1 (S. vaccaria)
MSPIFAHDVVNPQGLSFAFPAKDAENASSPV
SEQ ID NO: 10-sgd2a consensus cDNA (398 bp) encoding preSGD2 (S. vaccaria)
AGGGGAATGACACACAAAATAAATTCATACAAATCATTTATTTAGTCTCTAACTTACAAA
CTCCAATACTTCATTTGTGAAAATGTCTCCAATTTTTGCCCACGACGTAGTCAAGCCCCA
AGGCCTAAGTTTCGCTTTTCCGGCAAAAGATGCTGAAAATGCTTCATCCCCGGTGTAAAC
TTATGCCTGCAATGCGCTTCTGCGGCCTTTAGATACGATGTCTCCAGCCAAACCAAACCA
TAATGTCATGTCCGACGTTGTGTTTCTTACTTTTTTAGTTTTATTTTACGTTTATCGTTT
CGACTTTTAAGATGAAGAATAATGTATTTTGTTTATGGTTTGTAATAAAATTTAAAGGCC
GCTTTAGTGTACGTAAATTTATGGTTTTGTTTCCGGCC
SEQ ID NO: 11-preSGD2-linear polypeptide (31 aa) encoded by sgd2a (S. vaccaria)
MSPIFAHDVVKPQGLSFAFPAKDAENASSPV
SEQ ID NO: 12-preSGD3-linear polypeptide (31 aa) (S. vaccaria)
MSPILAHDVVKPQGLSFAFPAKDAENASSPV
SEQ ID NO: 13-Segetalin D-cyclic polypeptide (5 aa) from preSGD1, preSGD2 or
preSGD3 cyclization (S. vaccaria)
GLSFA
SEQ ID NO: 14-sgf1a-consensus cDNA (425 bp) encoding preSGF1 (S. vaccaria)
GCTGAAACCACAAATTAAAGCACAAACATAATCACCGATAATTTTACAAACATACATATT
ATCGTTCAATTCTTATCGTACATTATTATTATTATTGCAAGAATGGCCACCTCTTTCCAA
TTTGATGGTCTTAAGCCATCTTTTTCTGCTTCGTACAGCAGCAAGCCCATTCAAACTCAG
GTTTCAAACGGCATGGACAATGCTTCTGCCCCAGTGTAAACGCATCTAGCTAATGTCCGA
AATAAATGGCCTTTACTAGCTATAGACTCGACGTCGAGTTAATAAATCGTATACGATGGT
GCCTCATGTATCTCACTATTGTACTCGATCATCAACTCGTCGTTATGTCATTTGTGTGTA
ATCTTTATAATAAAATAAATAAATAAACAAAGTCTTTTGGTGAGTAAGTTCAAGACTTTT
AACTG
SEQ ID NO: 15-preSGF1-linear polypeptide (38 aa) encoded by sgf1a (S. vaccaria)
MATSFQFDGLKPSFSASYSSKPIQTQVSNGMDNASAPV
SEQ ID NO: 16-Segetalin F-cyclic polypeptide (9 aa) from preSGF1 cyclization (S.
vaccaria)
FSASYSSKP
SEQ ID NO: 17-sgg1-consensus cDNA (395 bp) encoding preSGG1 (S. vaccaria)
GATGACAACACAAAATACATCCAAAAAAATTAATTTAGTCTCTAACTTACAAAGTCCAAA
ACTACTTTATTTGTGAAAATGTCTCCAATTTTCGTCCACGAGGTGGTGAAGCCCCAAGGC
GTGAAATATGCTTTTCAGCCAAAAGATTCTGAAAATGCTTCAGCTCCAGTGTAAACTTAC
GCATGCAATGCGCTTCTACGGCCTTTAGATACGATGTCTCCGACCAAACCAAACAATAAT
CTTATGTCAAGTGTTGTATTACCCGTTTCTGTAATTTTATTTTATGTCTATTGTTTCGAC
TTTTAAGTTGAACTATGTACCCTAATTATGATGGTTTGTAATAAAATTTAAAGGCCATTT
TAATGTACGTAAATTTACACATTTTTCTTTTGTTC
SEQ ID NO: 18-preSGG1-linear polypeptide (31 aa) encoded by sgg1 (S. vaccaria)
MSPIFVHEVVKPQGVKYAFQPKDSENASAPV
SEQ ID NO: 19-Segetalin G-cyclic polypeptide (5 aa) from preSGG1 cyclization (S.
vaccaria)
GVKYA
SEQ ID NO: 20-sgh1 consensus cDNA (400 bp) encoding preSGH1 (S. vaccaria)
GATGACGCACAAAAACACATCCATACAAATCATTTATTTAGTCTTTAACTTACAAACTTC
AAAACTACTTTATTTGTGAAAATGTCTCCAATTTTTGCGCACGACATAGTCAAGCCCAAA
GGCTACAGATTTAGTTTTCAGGCAAAAGATGCTGAAAATGCTTCAGCCCCGGTGTAAACT
TATGTATGCAATGCACTTCTGCGGCTTTTAGATACGATGTCTCCAGCCAAATCAAAAACC
CTAATGTCATATCCAATGTCGTGTTTCTTATTTCTGTAGTTTTATTTTATGTTTATCGTT
TCGACTTTTAAGTTGAAGATGATGTACTTTGTTTATGATTTGTAATAAAATTTAAAAGCC
GTATTAGTGTACGTAAATTTACGATTTTCTTTTCGTTTAA
SEQ ID NO: 21-preSGH1-linear polypeptide (31 aa) encoded by sgh1 (S. vaccaria)
MSPIFAHDIVKPKGYRFSFQAKDAENASAPV
SEQ ID NO: 22-preSGH2-linear polypeptide (31 aa) (S. vaccaria)
MSPIFAHDIVKPKGYRFSFQAKDAENASSPV
SEQ ID NO: 23-Segetalin H-cyclic polypeptide (5 aa) from preSGH1 or preSGH2
cyclization (S. vaccaria)
GYRFS
SEQ ID NO: 24-grvka1-consensus cDNA (360 bp) encoding preGRVKA1 (S. vaccaria)
GATCACACAAAACATCCAAACAAATCATTTTAGTCTCTTAACTTAATTACGTACAGTCCA
TTACTGAAAATGTCTCCAATTTTAGCCCTCGACAGATACAAGCCCGAAGGCCGTGTGAAG
GCTTTTCAGGCAAAAGATGCTGAAAATGCTTCAGCCCCAGTCTAAACGTACGTTTGCGAT
GCGTTTTTGTGGTCTTTAGATACGATGCCTCCAACCAAACCATAATGTTATGTTCAATGT
TGTGTTTCTTATTTTGTAATTTTATTTTACGTGTATTATTTTGACTTTTAAAGTTGAATA
ATGTACCTCGTTTATGGTTTGTAATAAAAATCTAAAGGCCATTTTAGTGTTACAAAATTT
SEQ ID NO: 25-preGRVKA1-linear polypeptide (31 aa) encoded by grvka1 (S.
vaccaria)
MSPILALDRYKPEGRVKAFQAKDAENASAPV
SEQ ID NO: 26-Segetalin GRVKA1-cyclic polypeptide (5 aa) from preGRVKA1
cyclization (S. vaccaria)
GRVKA
SEQ ID NO: 27-glpgwp1-consensus cDNA (384 bp) encoding preGLPGWP1 (S.
vaccaria)
GACACACAAAAAACATTCAAACAAATCATTTAATCTCTAACTTTACAAGTTCAATACTTT
ATTTGTGAAAATGTCTCCAATCCTCTCCCACGACGTAGTCAAGCCCCAAGGTCTCCCTGG
TTGGCCTTTTCAGGCAAAAGATGTTGAAAATGCTTCAGCCCCTGTGTAAATTAATGCACA
GAATGCGCTTCTTCGGCCTTTAGATACGATGTTTCCAACCAAAATAAACCATAATGTTAT
GTCGAGTGTCGTGTTTTTTATTTCTGTAATTTATTTATGTGTATTGTTTCAATTTTTAAA
TTGAAACAATAAACTATTTTAATTGGTTTGTAATAAAATCTAAAAGGCCGTTTTAGCGTA
AATTTATGCATTCAACTGTCGTCT
SEQ ID NO: 28-preGLPGWP1-linear polypeptide (32 aa) encoded by glpgwp1 (S.
vaccaria)
MSPILSHDVVKPQGLPGWPFQAKDVENASAPV
SEQ ID NO: 29-Segetalin GLPGWP-cyclic polypeptide (6 aa) from preGLPGWP1
cyclization (S. vaccaria)
GLPGWP
SEQ ID NO: 30-fgthflpap1-consensus cDNA (435 bp) encoding preFGTHFLPAP1 (S.
vaccaria)
AAACCTGAAACCTCAAACCTCAAACCACAAACATATCATATCCTATATAAATTACCGTGA
AATCATTATTATTGCGAGAATGGCCACCTCTTTCCAACTTGATGGTCTTAAGCCTTCTTT
TGGTACGCACGGCCTGCCCGCGCCGATTCAGGTTCCAAACGGCATGGACGATGCTTGTGC
CCCAATGTAGATTCATTTAGCGTCTACAATAAATAAATGGCCTTTACTAGCTTTAGACTT
GAAGTCCCCAGAGTAATATTGTGTTACGTTTAGAGTTGTTTTATTGTTGTTTACTTGCAC
TGGACGTCGAGTTAAATCGTACACGATGGTCTCTTATGTATCTCACCACTGTACTTGATA
ATCAACTCCTCCTCCTGTCAATTGTGTGTTTACTTTCTATAAGTCAATAATAAAGAGTAA
AGGCATCTTTTCTCC
SEQ ID NO: 31-preFGTHFLPAP1-linear polypeptide (36 aa) encoded by fgthfipap1 (S.
vaccaria)
MATSFQLDGLKPSFGTHGLPAPIQVPNGMDDACAPM
SEQ ID NO: 32-Segetalin FGTHFLPAP-cyclic polypeptide (9 aa) from preFGTHFLPAP
cyclization (S. vaccaria)
FGTHFLPAP
SEQ ID NO: 33-Flax (Bethune) cp1-cDNA (660 bp) (Linum usitatissimum cultivar
Bethune)
ATGGCTGCTGCTTCCTCTCTCGCTCTGGCCACCGCTAGCCTAGTTGCTACCGGCGCCGGC
GGCCGTAATAACGCCTTCCTACCCTCGAAGAACAAGACACCAAACCTTTTCCTTAATCCC
AACAAAACAACGTCGTCAACAGTGAAAGCTGTTGTCTCATCATCATCATGCAAACGCCCC
TACCCGAAAGGAGATGCTAGTTTATTCTTGGGTATTGATGATGTATTCGGAAAGGATGCT
GTTGCTGGCCATGATAATGATCAGGATGCTGCAAGTGGCCAGGAGATGGCCGCCGATGAT
ATGTTGATGCCATTCTTTTGGATATTCGGAAAAGAAGGACAGCAGCAGGAGGCCGAGGAG
AGCAGCGATGATATGTTGATGCCATTCTTTTGGATATTCGGCAAGGAAGGACAGCAGCAG
GAGGCCGAGAGCAGCGATGATATGTTGCTGCCATTCTTTTGGATATTCGGCAAGGAAGGA
CAGCAGCAGGAGGCCGAGAGCAGCGATGATATGCTGATGCCTTTCTTTTGGATATTCGGC
AAGCAGCAGCAGCAGCAGGGTGAGAGCAGCGATGATATGTTGATGCCTTTCTTTTGGGTA
TTCGGCAAGCAAGGTGACAACAACAAGGGCGATGCTGTAGAAGCAATCCTTAAGAACTAG
SEQ ID NO: 34-Flax (Bethune) cp1-genomic (1602 bp) (Linum usitatissimum cultivar
Bethune)
ATGGCTGCTGCTTCCTCTCTCGCTCTGGCCACCGCTAGCCTAGTTGCTACCGGCGCCGGC
GGCCGTAATAACGCCTTCCTACCCTCGAAGAACAAGACACCAAACCTTTTCCTTAATCCC
AACAAAACAACGTCGTCAACAGTGAAAGCTGTTGTCTCATCATCATCATGCAAACGCCCC
TACCCGAAAGGAGATGCTAGTTTATTCTTGGGTATTGATGATGTATTCGGAAAGGATGCT
GTTGCTGGCCATGATAATGATCAGGATGGTTTGTTGTTTCCACTCTTGCTTTTTATATTG
GGGATGGCGAGAACAAGGTGTAGGAAATTGTTTAGATATCGTTTAGATGCATATTAACTA
ATCCCATCATTATATCTAACTTTCTTATATCTTTCTTATATAAATCAATAACTTTCTTAT
ATAAATCAATAACAAAGGTTTTTAGTACTAATCAATGATTAGTATTTGCTGAAGCCTTTG
GTTTAATGACTAGTACTTGCTGAAGCCTTTAGATTGATTACGACTTGTGAGAATTTCATG
TGTAGCTTCTTTTTTCAGTTTACGCTAATTGGATTTTGGATTTTCTTTGTCAATACTGGC
TAAAACGTTTGATCGAAAAACGATTTATCAAAGTATTTGGTAATTAGGGTTTTCTTTTAA
AAGTTTTTAATGGCTTCCTAATTCAGTTTTAGATAAACTATTACAACTAACCATCAATTT
TGGATAAACTATTACAACTAACCATCAATTTTAGATAAACTATTACAACTAACCATCAGT
TGTAGATAAACTATTACAACTAACCATCAGTTGTAGATAAACTATTACAACTAACCATCA
GTTGTAGATAAACTATTACAACTAACCATCAGTTGTAGATAAACTATTACAACTAACCCT
CTATTTATAGAATTTCTCATAAACTTTCACCCTATTTGACCATCAACTCATTAAGCTAAT
CCATTTACATTAATCCGGTCCATACTACTAAAAAAGTGTGTGTCCATATTACTAAAAAAG
CGTGTGAAAGTGTGTGACTTTGTAGGACCCGATTCGATTAGTCGTGGTCCAAACTACTAA
TTAACATTGACCTCTAATAAGATGTGTTAACTCCTAACTGGACCGAATTACTTTTGATTA
ATCAGCCTCCCTAGTTTTTATTCGGATTCGGATTTAGGCCGAAGGACATAAATTCTTCAC
AATGATGCAGCTGCAAGTGGCCAGGAGATGGCCGCCGATGATATGTTGATGCCATTCTTT
TGGATATTCGGAAAAGAAGGACAGCAGCAGGAGGCCGAGGAGAGCAGCGATGATATGTTG
ATGCCATTCTTTTGGATATTCGGCAAGGAAGGACAGCAGCAGGAGGCCGAGAGCAGCGAT
GATATGTTGCTGCCATTCTTTTGGATATTCGGCAAGGAAGGACAGCAGCAGGAGGCCGAG
AGCAGCGATGATATGCTGATGCCTTTCTTTTGGATATTCGGCAAGCAGCAGCAGCAGCAG
GGTGAGAGCAGCGATGATATGTTGATGCCTTTCTTTTGGGTATTCGGCAAGCAAGGTGAC
AACAACAAGGGCGATGCTGTAGAAGCAATCCTTAAGAACTAG
SEQ ID NO: 35-Flax (Bethune) CP1-linear polypeptide (219 aa) encoded by Flax
(Bethune) cp1 cDNA (Linum usitatissimum cultivar Bethune)
MAAASSLALATASLVATGAGGRNNAFLPSKNKTPNLFLNPNKTTSSTVKAVVSSSSCKRP
YPKGDASLFLGIDDVFGKDAVAGHDNDQDAASGQEMAADDMLMPFFWIFGKEGQQQEAEE
SSDDMLMPFFWIFGKEGQQQEAESSDDMLLPFFWIFGKEGQQQEAESSDDMLMPFFWIFG
KQQQQQGESSDDMLMPFFWVFGKQGDNNKGDAVEAILKN
SEQ ID NO: 36-Flax (Bethune) CP1-linear polypeptide (511 aa) encoded by Flax
(Bethune) cp1 genomic DNA (Linum usitatissimum cultivar Bethune)
MAAASSLALATASLVATGAGGRNNAFLPSKNKTPNLFLNPNKTTSSTVKAVVSSSSCKRP
YPKGDASLFLGIDDVFGKDAVAGHDNDQDGLLFPLLLFILGMARTRCRKLFRYRLDAYLI
PSLYLTFLYLSYINQLSYINQQRFLVLINDYLLKPLVLVLAEAFRLITTCENFMCSFFFQ
FTLIGFWIFFVNTGNVSKNDLSKYLVIRVFFKFLMASFSFRTITTNHQFWINYYNPSILD
KLLQLTISCRTITTNHQLINYYNPSVVDKLLQLTISCRTITTNPLFIEFLINFHPIPSTH
ANPFTLIRSILLKKCVSILLKKRVKVCDFVGPDSISRGPNYLTLTSNKMCLLTGPNYFLI
SLPSFYSDSDLGRRTILHNDAAASGQEMAADDMLMPFFWIFGKEGQQQEAEESSDDMLMP
FFWIFGKEGQQQEAESSDDMLLPFFWIFGKEGQQQEAESSDDMLMPFFWIFGKQQQQQGE
SSDDMLMPFFWVFGKQGDNNKGDAVEAILKN
SEQ ID NO: 37-MLMPFFWI-cyclic peptide (8 aa) from Flax (Bethune) CP1 cyclization
(Linum usitatissimum cultivar Bethune)
MLMPFFWI
SEQ ID NO: 38-MLLPFFWI-cyclic peptide (8 aa) from Flax (Bethune) CP1 cyclization
(Linum usitatissimum cultivar Bethune)
MLLPFFWI
SEQ ID NO: 39-MLMPFFWV-cyclic peptide (8 aa) from Flax (Bethune) CP1 cyclization
(Linum usitatissimum cultivar Bethune)
MLMPFFWV
SEQ ID NO: 40-linear polypeptide (48 aa) encoded by cDNA of Genbank DN798249
(Citrus paradise)
MKTLAGAGMSDPSEGLVLPSSIADDDVGNDNLDLIVIPQYGRNPDYYG
SEQ ID NO: 41-GLVLPS-cyclic polypeptide (6 aa) from cyclization of linear polypeptide
encoded by cDNA of Genbank DN798249 (Citrus paradise)
GLVLPS
SEQ ID NO: 42-linear polypeptide (48 aa) encoded by cDNA of Genbank EG026628
(Citrus clementina)
METTCAGNNWSEGLLLPPFGSIADDDVMNDNLDFLNVPQYGRNPDYMG
SEQ ID NO: 43-GLLLPPFG-cyclic polypeptide (8 aa) from cyclization of linear
polypeptide encoded by cDNA of Genbank EG026628 (Citrus clementina)
GLLLPPFG
SEQ ID NO: 44-linear polypeptide (49 aa) encoded by cDNA of Genbank DC900394
(Citrus sinensis cDNA clone VS28967)
MKTLPGAGMSDPSEGYLLPPSSIADDDVGNDNLDLIVIPQYGRNPDYYG
SEQ ID NO: 45-GYLLPPS-cyclic polypeptide (7 aa) from cyclization of linear
polypeptide encoded by cDNA of Genbank DC900394 (Citrus sinensis cDNA clone
VS28967)
GYLLPPS
SEQ ID NO: 46-linear polypeptide (33 aa) encoded by cDNA of Genbank AW697819
(Dianthus caryophyllus cDNA clone HM002)
MSPNSTRDILKPQGPIPFYGFQAKDAENASVPV
SEQ ID NO: 47-GPIPFYG-cyclic polypeptide (7 aa) from cyclization of linear
polypeptide encoded by cDNA of Genbank AW697819 (Dianthus caryophyllus cDNA
clone HM002)
GPIPFYG
SEQ ID NO: 48-linear polypeptide (32 aa) encoded by cDNA of Genbank AW697902
(Dianthus caryophyllus cDNA clone HM085)
MSPNSTLDILKPLGLPYEQFQAKDSENASAPV
SEQ ID NO: 49-GLPYEQ-cyclic polypeptide (6 aa) from cyclization of linear
polypeptide encoded by cDNA of Genbank AW697902 (Dianthus caryophyllus cDNA
clone HM085)
GLPYEQ
SEQ ID NO: 50-linear polypeptide (32 aa) encoded by cDNA of Genbank CF259529
(Dianthus caryophyllus cDNA clone Dc080)
MSPNSTRDLLKPLGYKDCCFQAKDLENAAVPV
SEQ ID NO: 51-GYKDCC-cyclic polypeptide (6 aa) from cyclization of linear
polypeptide encoded by cDNA of Genbank CF259529 (Dianthus caryophyllus cDNA
clone Dc080)
GYKDCC
SEQ ID NO: 52-Primer JC1 (19 bp)
CACCATGTCTCCAATCCTC
SEQ ID NO: 53-Primer JC2 (18 bp)
TTACACAGGGGCTGAAGC
SEQ ID NO: 54-Primer JC3 (21 bp)
CCGACAGTGGTCCCAAAGATG
SEQ ID NO: 55-Primer JC4 (20 bp)
GCCTGAAAAGCCCAAACTGG
SEQ ID NO: 56-Primer CP1-F (32 bp)
GCGGCCGCATGGCTGCTGCTTCCTCTCTCGCT
SEQ ID NO: 57-Primer CP1-R1 (38 bp)
CCTGCAGGCTAGTTCTTAAGGATTGCTTCTACAGCATC
SEQ ID NO: 58-CP1 Linker (38 bp)
GCGGCCGCAAAAAACCTGCAGGACCCGGGAGGCGCGCC

### REFERENCES

### The contents of the Entirety of Each of Which Are Incorporated by this Reference

Altschul S F, Gish W, Miller W, Myers E W, Lipman D J. (1990) Basic local alignment search tool. Journal of Molecular Biology. 215: 403-410.
Alvarez J P, Pekker I, Goldshmidt A, Blum E, Amsellem Z, Eshed Y. (2006) Endogenous and synthetic microRNAs stimulate simultaneous, efficient, and localized regulation of multiple targets in diverse species. Plant Cell. 8: 1134-51.
Balsevich J J, Bishop G G, Deibert L K. (2009) Phytochem Anal. 20: 38-49.
Bechtold N, Ellis J, Pellefer G. (1993) In planta Agrobacterium-mediated gene transfer by infiltration of adult Arabidopsis thaliana plants. C.R. Acad. ScL Ser. III Sci. Vie, 316: 1194-1199.
Becker D, Brettschneider R, Lorz H. (1994) Fertile transgenic wheat from microprojectile bombardment of scutellar tissue. Plant J. 5: 299-307.
Chou H-H, Holmes M H. (2001) DNA sequence quality trimming and vector removal. Bioinformatics. 17: 1093-1104.
Craik D J, et al. (2004) Curr. Protein Pept. Sci. 5: 297-315.
Dahiya R, et al. (2007a) Arch Pharm Res. 30(11): 1380-1386.
Dahiya R. (2007b) Acta Polonise Pharmaceutica—Drug Research. 64(6): 509-516.
Dahiya R, et al. (2008a) Synthesis and pharmacological investigation of Segetalin C as a novel anti-fungal and cytotoxic agent. Arzneimittel Hel-Forschung (Drug Research). 58(1): 29-34
Dahiya R, and Kumar. (2008b) Synthetic and biological studies on a cyclopeptide of plant origin. Journal of Zhejiang Univ Sci B. 9(5): 391-400.
Datla R, Anderson J W, Selvaraj G. (1997) Plant promoters for transgene expression. Biotechnology Annual Review. 3: 269-296.
Davies J S. (1999) Cyclic, Modified and Conjugated Peptides. In Amino Acids, Peptides and Proteins. Vol. 30, Chapter 4, pp 285-334.
Davies J S. (2007) The Cyclization of Peptides and Depsipeptides. J. Peptide Sci. 9: 471-501.
DeBlock M, DeBrouwer D, Tenning P. (1989) Transformation of Brassica napus and Brassica oleracea using Agrobacterium tumefaciens and the expression of the bar and neo genes in the transgenic plants. Plant Physiol. 91: 694-701.
Depicker A, Montagu M V. (1997) Post-transcriptional gene silencing in plants. Curr Opin Cell Biol. 9: 373-82.
Donia M S, Hathaway B J, Sudek S, Haygood M G, Rosovitz M J, Ravel J, Schmidt E W. (2006) Natural combinatorial peptide libraries in cyanobacterial symbionts of marine ascidians. Nature Chemical Biology. 2(12): 729-735.
Ewing B, Hillier L, Wendl M C, Green P. (1998) Base-calling of automated sequencer traces using phred I. Accuracy assessment. Genome Res. 8: 175-185.
Ferrie A M R, Mykytyshyn M, Bethune T. (2006) Methods for Producing Microspore Derived Doubled Haploid Apiaceae. International Patent Publication WO 2006/125310 published Nov. 30, 2006.
Gruber C, et al. (2008) Distribution and evolution of circular miniproteins in flowering plants. The Plant Cell. 20: 2471-2483.
Grunewald J, Marahiel M A. (2006) Chemoenzymatic and template-directed synthesis of bioactive macrocyclic peptides. Microbiology and Molecular Biology Reviews. 70: 121-146.
Helliwell C A, Waterhouse P M. (2005) Constructs and methods for hairpin RNA-mediated gene silencing in plants. Methods Enzymology 392: 24-35.
Henikoff S, Till B J, Comai L. (2004) TILLING. Traditional mutagenesis meets functional genomics. Plant Physiol. 135: 630-6.
Huang X, Madan A. (1999) CAPS: A DNA sequence assembly program. Genome Res. 9: 868-877.
Karimi M, Inze D, Depicker A. (2002) GATEWAY vectors for Agrobacterium-mediated plant transformation. Trends Plant Sci. 7(5): 193-195.
Katavic Y, Haughn G W, Reed D, Martin M, Kunst L. (1994) In planta transformation of Arabidopsis thaliana. Mol. Gen. Genet. 245: 363-370.
Li X, Song Y, Century K, Straight S, Ronald P, Dong X, Lassner M, Zhang Y. (2001) A fast neutron deletion mutagenesis-based reverse genetics system for plants. Plant J. 27: 235-242.
Meesapyodsuk D, Balsevich J, Reed D W, Covello P S. (2007) Saponin biosynthesis in Saponaria vaccaria. cDNAs encoding beta-amyrin synthase and a triterpene carboxylic acid glucosyltransferase. Plant Physiol. 143 (2):959-969.
Meyer P. (1995) Understanding and controlling transgene expression. Trends in Biotechnology. 13: 332-337.
Miller R T, Christoffels A G, Gopalakrishnan C, Burke J, Ptitsyn A A, Broveak T R, Hide W A. (1999) A comprehensive approach to clustering of expressed human gene sequence: the sequence tag alignment and consensus knowledge base. Genome Res. 9(11): 1143-1155.
Moloney M M, Walker J M, Sharma K K. (1989) High efficiency transformation of Brassica napus using Agrobacterium vectors. Plant Cell Rep. 8: 238-242.
Morita H, et al. (1995a) Tetrahedron. 51: 5987-6002.
Morita H, et al. (1995b) Tetrahedron. 51: 6003-6014.
Morita H. et al. (1996) Phytochemistry. 42: 439-441.
Morita H, et al. (1997) Bioorg. Med. Chem. 5(11): 2063-2067.
Morita H, et al. (2006) Structure of a new cyclic nonapeptide, segetalin F, and vasorelaxant activity of segetalins from Vaccaria segetalis. Bioorganic & Medicinal Chemistry Letters. 16(17): 4458-4461.
Morita H, et al. (2007) Bioorganic & Medicinal Chemistry Letters. 17: 5410-5413.
Neddleman and Wunsch. (1970) J. Mol. Biol. 48: 443.
Nehra N S, Chibbar R N, Leung N, Caswell K, Mallard C, Steinhauer L, Baga M, Kartha K K. (1994) Self-fertile transgenic wheat plants regenerated from isolated scutellar tissues following microprojectile bombardment with two distinct gene constructs. Plant J. 5: 285-297.
Pearson and Lipman. (1988) Proc. Natl. Acad. Sci. (U.S.A.) 85: 2444.
Picur B, et al. (2007) J. Pept. Sci. 12(9): 569-574.
Potrykus L. (1991) Gene transfer to plants: Assessment of publish approaches and results. Annu. Rev. Plant Physiol. Plant Mol. Biol. 42: 205-225.
Rhodes C A, Pierce D A, Mettler I J, Mascarenhas D, Detmer J J. (1988) Genetically transformed maize plants from protoplasts. Science. 240: 204-207.
Sambrook J, Fritsch E F, Maniatis T. (1989) Molecular Cloning: A Laboratory Manual 2^{nd} edn. Cold Spring Harbor: Cold Spring Harbor Laboratory Press.
Sambrook J, Fritsch E F, Maniatis T. (2001) Molecular Cloning: A Laboratory Manual 3^{rd} edn. Cold Spring Harbor: Cold Spring Harbor Laboratory Press.
Sanford J C, Klein T M, Wolf E D, Allen N. (1987) Delivery of substances into cells and tissues using a particle bombardment process. J. Part. Sci. Technol. 5: 27-37.
Sarabia F, et al. (2004) Curr. Med. Chem. 11: 1309-1332.
Schmidt J F, Moore M D, Pelcher L E, Covello P S. (2007) High efficiency Agrobacterium rhizogenes-mediated transformation of Saponaria vaccaria L. (Caryophyllaceae) using fluorescence selection. Plant Cell Reports. 26: 1547-1554.
Schwab R, Ossowski S, Riester M, Warthmann N, Weigel D. (2006) Highly specific gene silencing by artificial microRNAs in Arabidopsis. Plant Cell, 18: 1121-33.
Scott C P, Abel-Santos E, Wall M, Wahnon D C, Benkovic S J. (1999) Production of Cyclic Peptides and Proteins in vivo. PNAS. 96(24): 13638-13643.
Seiber S A, Marahiel M A. (2003) Learning from nature's drug factories: non-ribosomal synthesis of macrocyclic peptides. Journal of Bacteriology. 185: 7036-7043.
Shimamoto K, Terada R, Izawa T, Fujimoto H. (1989) Fertile transgenic rice plants regenerated from transformed protoplasts. Nature. 335: 274-276.
Smith and Waterman. (1981) Ad. App. Math. 2: 482.
Songstad D D, Somers D A, Griesbach R J. (1995) Advances in alternative DNA delivery techniques. Plant Cell, Tissue and Organ Culture. 40: 1-15.
Stam M, de Bruin R, van Blokland R, van der Hoorn R A, Mol J N, Kooter J M. (2000) Distinct features of post-transcriptional gene silencing by antisense transgenes in single copy and inverted T-DNA repeat loci. Plant J. 21: 27-42.
Tan N-H, Zhou J. (2006) Plant Cyclopeptides. Chem. Rev. 106: 840-895.
Teerawanichpan P, et al. (2007) Biochimica et Biophysica Acta. 1770:1360-1368.
Vasil I K. (1994) Molecular improvement of cereals. Plant Mol. Biol. 5: 925-937.
Walden R, Wingender R. (1995) Gene-transfer and plant regeneration techniques. Trends in Biotechnology. 13: 324-331.
Yun Y S, et al. (1997) J. Nat. Prod. 60(3): 216-218.

Other advantages that are inherent to the structure are obvious to one skilled in the art. The embodiments are described herein illustratively and are not meant to limit the scope of the invention as claimed. Variations of the foregoing embodiments will be evident to a person of ordinary skill and are intended by the inventor to be encompassed by the following claims.

1. A method of producing a cyclopeptide comprising:
providing a linear polypeptide comprising the amino acid sequence as set forth in SEQ ID NO: 3, SEQ ID NO: 7, SEQ ID NO: 13, SEQ ID NO: 16, SEQ ID NO: 19, SEQ ID NO: 23, SEQ ID NO: 26, SEQ ID NO: 29, SEQ ID NO: 32, SEQ ID NO: 37, SEQ ID NO: 38, SEQ ID NO: 39, SEQ ID NO: 41, SEQ ID NO: 43, SEQ ID NO: 45, SEQ ID NO: 47, SEQ ID NO: 49 or SEQ ID NO: 51; and, subjecting the linear polypeptide to conditions under which a cyclopeptide consisting of the amino acid sequence as set forth in SEQ ID NO: 3, SEQ ID NO: 7, SEQ ID NO: 13, SEQ ID NO: 16, SEQ ID NO: 19, SEQ ID NO: 23, SEQ ID NO: 26, SEQ ID NO: 29, SEQ ID NO: 32, SEQ ID NO: 37, SEQ ID NO: 38, SEQ ID NO: 39, SEQ ID NO: 41, SEQ ID NO: 43, SEQ ID NO: 45, SEQ ID NO: 47, SEQ ID NO: 49 or SEQ ID NO: 51 is produced by cyclization of the linear polypeptide.

2. The method according to claim 1, wherein the linear polypeptide comprises the amino acid sequence as set forth in SEQ ID NO: 2, SEQ ID NO: 5, SEQ ID NO: 6, SEQ ID NO: 9, SEQ ID NO: 11, SEQ ID NO: 12, SEQ ID NO: 15, SEQ ID NO: 18, SEQ ID NO: 21, SEQ ID NO: 22, SEQ ID NO: 25, SEQ ID NO: 28, SEQ ID NO: 31, SEQ ID NO: 35, SEQ ID NO: 36, SEQ ID NO: 40, SEQ ID NO: 42, SEQ ID NO: 44, SEQ ID NO: 46, SEQ ID NO: 48, SEQ ID NO: 50, or an amino acid sequence thereof having a conservative substitution.

3. The method according to claim 1, wherein the linear polypeptide is provided by transforming a host cell, tissue or organism with a means for encoding the linear polypeptide.

4. The method according to claim 3, wherein the means for encoding the linear polypeptide comprises a nucleic acid molecule having a nucleotide sequence having at least 80% sequence identity to the nucleotide sequence as set forth in SEQ ID NO: 1, SEQ ID NO: 4, SEQ ID NO: 8, SEQ ID NO: 10, SEQ ID NO: 14, SEQ ID NO: 17, SEQ ID NO: 20, SEQ ID NO: 24, SEQ ID NO: 27, SEQ ID NO: 30, SEQ ID NO: 33, SEQ ID NO: 34, a codon degenerate sequence thereof or a full length complement thereof.

5. The method according to claim 3, wherein the cell, tissue or organism is of a plant species that naturally produces cyclopeptides and the conditions under which the linear polypeptide is cyclized are provided by the host cell, tissue or organism.

6. The method according to claim 5, wherein the cell, tissue or organism is roots of a plant.

7. The method according to claim 5, wherein the plant species is of genus Saponaria.

8. The method according to claim 3, wherein the means for encoding the linear polypeptide comprises the nucleotide sequence as set forth in SEQ ID NO: 1.

9. A method of reducing cyclopeptide content in a host cell, tissue or plant comprising: reducing expression in the cell, tissue or plant of a nucleic acid molecule comprising a nucleotide sequence having at least 80% sequence identity to the nucleotide sequence as set forth in SEQ ID NO: 1, SEQ ID NO: 4, SEQ ID NO: 8, SEQ ID NO: 10, SEQ ID NO: 14, SEQ ID NO: 17, SEQ ID NO: 20, SEQ ID NO: 24, SEQ ID NO: 27, SEQ ID NO: 30, SEQ ID NO: 33, SEQ ID NO: 34, a codon degenerate sequence thereof or a full length complement thereof, compared to expression of the nucleotide sequence in the cell, tissue or plant before expression was reduced.

10. An isolated nucleic acid molecule comprising a nucleotide sequence having at least 90% sequence identity to the nucleotide sequence as set forth in SEQ ID NO: 1, SEQ ID NO: 4, SEQ ID NO: 8, SEQ ID NO: 10, SEQ ID NO: 14, SEQ ID NO: 17, SEQ ID NO: 20, SEQ ID NO: 24, SEQ ID NO: 27, SEQ ID NO: 30, SEQ ID NO: 33, SEQ ID NO: 34, a codon degenerate sequence thereof, or a full length complement thereof.

11. The isolated nucleic acid molecule according to claim 10 having 100% sequence identity to the nucleotide sequence as set forth in SEQ ID NO: 1, SEQ ID NO: 4, SEQ ID NO: 8, SEQ ID NO: 10, SEQ ID NO: 14, SEQ ID NO: 17, SEQ ID NO: 20, SEQ ID NO: 24, SEQ ID NO: 27, SEQ ID NO: 30, SEQ ID NO: 33, SEQ ID NO: 34, a codon degenerate sequence thereof, or a full length complement thereof.

12. An isolated nucleic acid molecule comprising the nucleotide sequence flanking a cyclopeptide encoding region of the nucleotide sequences as defined in claim 10.

13. A nucleic acid construct comprising one or more of the nucleic acid molecules as defined in claim 10 operatively linked to one or more nucleotide sequences for aiding in transformation of a cell with the construct.

14. An isolated linear polypeptide comprising the amino acid sequence as set forth in SEQ ID NO: 2, SEQ ID NO: 5, SEQ ID NO: 6, SEQ ID NO: 9, SEQ ID NO: 11, SEQ ID NO: 12, SEQ ID NO: 15, SEQ ID NO: 18, SEQ ID NO: 21, SEQ ID NO: 22, SEQ ID NO: 25, SEQ ID NO: 28, SEQ ID NO: 31, SEQ ID NO: 35 or SEQ ID No: 36, or an amino acid sequence thereof having a conservative substitution.

15. An isolated cyclopeptide consisting of the amino acid sequence as set forth in SEQ ID NO: 26, SEQ ID NO: 29, SEQ ID NO: 32, SEQ ID NO: 47, SEQ ID NO: 49 or SEQ ID NO: 51.

16. (canceled)

17. A method of identifying a gene or polypeptide related to cyclopeptide production comprising:
selecting a nucleic acid molecule that is known to encode a reference cyclopeptide;
identifying a flanking sequence in the nucleic acid molecule or in a linear polypeptide encoded by the nucleic acid molecule, the flanking sequence flanking a nucleotide sequence of the nucleic acid molecule that encodes the reference cyclopeptide or flanking an amino acid sequence of the linear polypeptide that corresponds to the reference cyclopeptide;
searching a database of nucleic acid molecules or polypeptides for target sequences that have at least 80% sequence identity to the flanking sequence to thereby identify nucleotide or amino acid sequences that correspond to the gene or polypeptide related to cyclopeptide production.

18. The method according to claim 17, wherein the target sequence has at least 95% sequence identity to the flanking sequence.
